SEQUENCE LISTING (7 sequences)

SEQ ID NO: 1 - protein, 315 aa, Artificial Sequence
  Description of Artificial Sequence Synthetic polypeptide
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 2 - protein, 25 aa, Artificial Sequence
  Description of Artificial Sequence Synthetic peptide
GDYKDDDDKDYKDDDDKDYKDDDDK

SEQ ID NO: 3 - protein, 1600 aa, Artificial Sequence
  Description of Artificial Sequence Synthetic polypeptide
  MISC_FEATURE (7)..(11): This region may encompass “SGGQQ,” “GAGQQ,” “GQGPY,” “AGQQ” or “SQ,” wherein some positions may be absent
  MISC_FEATURE (15)..(19): This region may encompass “SGGQQ,” “GAGQQ,” “GQGPY,” “AGQQ” or “SQ,” wherein some positions may be absent
  MISC_FEATURE (23)..(27): This region may encompass “SGGQQ,” “GAGQQ,” “GQGPY,” “AGQQ” or “SQ,” wherein some positions may be absent
  MISC_FEATURE (31)..(35): This region may encompass “SGGQQ,” “GAGQQ,” “GQGPY,” “AGQQ” or “SQ,” wherein some positions may be absent
  MISC_FEATURE (39)..(43): This region may encompass “SGGQQ,” “GAGQQ,” “GQGPY,” “AGQQ” or “SQ,” wherein some positions may be absent
  MISC_FEATURE (47)..(51): This region may encompass “SGGQQ,” “GAGQQ,” “GQGPY,” “AGQQ” or “SQ,” wherein some positions may be absent
  MISC_FEATURE (55)..(59): This region may encompass “SGGQQ,” “GAGQQ,” “GQGPY,” “AGQQ” or “SQ,” wherein some positions may be absent
  MISC_FEATURE (63)..(67): This region may encompass “SGGQQ,” “GAGQQ,” “GQGPY,” “AGQQ” or “SQ,” wherein some positions may be absent
  MISC_FEATURE (4)..(67): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (71)..(80): This region may encompass 6-10 residues
  MISC_FEATURE (87)..(91): This region may encompass “SGGQQ,” “GAGQQ,” “GQGPY,” “AGQQ” or “SQ,” wherein some positions may be absent
  MISC_FEATURE (95)..(99): This region may encompass “SGGQQ,” “GAGQQ,” “GQGPY,” “AGQQ” or “SQ,” wherein some positions may be absent
  MISC_FEATURE (103)..(107): This region may encompass “SGGQQ,” “GAGQQ,” “GQGPY,” “AGQQ” or “SQ,” wherein some positions may be absent
  MISC_FEATURE (111)..(115): This region may encompass “SGGQQ,” “GAGQQ,” “GQGPY,” “AGQQ” or “SQ,” wherein some positions may be absent
  MISC_FEATURE (119)..(123): This region may encompass “SGGQQ,” “GAGQQ,” “GQGPY,” “AGQQ” or “SQ,” wherein some positions may be absent
  MISC_FEATURE (127)..(131): This region may encompass “SGGQQ,” “GAGQQ,” “GQGPY,” “AGQQ” or “SQ,” wherein some positions may be absent
  MISC_FEATURE (135)..(139): This region may encompass “SGGQQ,” “GAGQQ,” “GQGPY,” “AGQQ” or “SQ,” wherein some positions may be absent
  MISC_FEATURE (143)..(147): This region may encompass “SGGQQ,” “GAGQQ,” “GQGPY,” “AGQQ” or “SQ,” wherein some positions may be absent
  MISC_FEATURE (84)..(147): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (151)..(160): This region may encompass 6-10 residues
  MISC_FEATURE (167)..(171): This region may encompass “SGGQQ,” “GAGQQ,” “GQGPY,” “AGQQ” or “SQ,” wherein some positions may be absent
  MISC_FEATURE (175)..(179): This region may encompass “SGGQQ,” “GAGQQ,” “GQGPY,” “AGQQ” or “SQ,” wherein some positions may be absent
  MISC_FEATURE (183)..(187): This region may encompass “SGGQQ,” “GAGQQ,” “GQGPY,” “AGQQ” or “SQ,” wherein some positions may be absent
  MISC_FEATURE (191)..(195): This region may encompass “SGGQQ,” “GAGQQ,” “GQGPY,” “AGQQ” or “SQ,” wherein some positions may be absent
  MISC_FEATURE (199)..(203): This region may encompass “SGGQQ,” “GAGQQ,” “GQGPY,” “AGQQ” or “SQ,” wherein some positions may be absent
  MISC_FEATURE (207)..(211): This region may encompass “SGGQQ,” “GAGQQ,” “GQGPY,” “AGQQ” or “SQ,” wherein some positions may be absent
  MISC_FEATURE (215)..(219): This region may encompass “SGGQQ,” “GAGQQ,” “GQGPY,” “AGQQ” or “SQ,” wherein some positions may be absent
  MISC_FEATURE (223)..(227): This region may encompass “SGGQQ,” “GAGQQ,” “GQGPY,” “AGQQ” or “SQ,” wherein some positions may be absent
  MISC_FEATURE (164)..(227): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (231)..(240): This region may encompass 6-10 residues
  MISC_FEATURE (247)..(251): This region may encompass “SGGQQ,” “GAGQQ,” “GQGPY,” “AGQQ” or “SQ,” wherein some positions may be absent
  MISC_FEATURE (255)..(259): This region may encompass “SGGQQ,” “GAGQQ,” “GQGPY,” “AGQQ” or “SQ,” wherein some positions may be absent
  MISC_FEATURE (263)..(267): This region may encompass “SGGQQ,” “GAGQQ,” “GQGPY,” “AGQQ” or “SQ,” wherein some positions may be absent
  MISC_FEATURE (271)..(275): This region may encompass “SGGQQ,” “GAGQQ,” “GQGPY,” “AGQQ” or “SQ,” wherein some positions may be absent
  MISC_FEATURE (279)..(283): This region may encompass “SGGQQ,” “GAGQQ,” “GQGPY,” “AGQQ” or “SQ,” wherein some positions may be absent
  MISC_FEATURE (287)..(291): This region may encompass “SGGQQ,” “GAGQQ,” “GQGPY,” “AGQQ” or “SQ,” wherein some positions may be absent
  MISC_FEATURE (295)..(299): This region may encompass “SGGQQ,” “GAGQQ,” “GQGPY,” “AGQQ” or “SQ,” wherein some positions may be absent
  MISC_FEATURE (303)..(307): This region may encompass “SGGQQ,” “GAGQQ,” “GQGPY,” “AGQQ” or “SQ,” wherein some positions may be absent
  MISC_FEATURE (244)..(307): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (311)..(320): This region may encompass 6-10 residues
  MISC_FEATURE (327)..(331): This region may encompass “SGGQQ,” “GAGQQ,” “GQGPY,” “AGQQ” or “SQ,” wherein some positions may be absent
  MISC_FEATURE (335)..(339): This region may encompass “SGGQQ,” “GAGQQ,” “GQGPY,” “AGQQ” or “SQ,” wherein some positions may be absent
  MISC_FEATURE (343)..(347): This region may encompass “SGGQQ,” “GAGQQ,” “GQGPY,” “AGQQ” or “SQ,” wherein some positions may be absent
  MISC_FEATURE (351)..(355): This region may encompass “SGGQQ,” “GAGQQ,” “GQGPY,” “AGQQ” or “SQ,” wherein some positions may be absent
  MISC_FEATURE (359)..(363): This region may encompass “SGGQQ,” “GAGQQ,” “GQGPY,” “AGQQ” or “SQ,” wherein some positions may be absent
  MISC_FEATURE (367)..(371): This region may encompass “SGGQQ,” “GAGQQ,” “GQGPY,” “AGQQ” or “SQ,” wherein some positions may be absent
  MISC_FEATURE (375)..(379): This region may encompass “SGGQQ,” “GAGQQ,” “GQGPY,” “AGQQ” or “SQ,” wherein some positions may be absent
  MISC_FEATURE (383)..(387): This region may encompass “SGGQQ,” “GAGQQ,” “GQGPY,” “AGQQ” or “SQ,” wherein some positions may be absent
  MISC_FEATURE (324)..(387): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (391)..(400): This region may encompass 6-10 residues
  MISC_FEATURE (407)..(411): This region may encompass “SGGQQ,” “GAGQQ,” “GQGPY,” “AGQQ” or “SQ,” wherein some positions may be absent
  MISC_FEATURE (415)..(419): This region may encompass “SGGQQ,” “GAGQQ,” “GQGPY,” “AGQQ” or “SQ,” wherein some positions may be absent
  MISC_FEATURE (423)..(427): This region may encompass “SGGQQ,” “GAGQQ,” “GQGPY,” “AGQQ” or “SQ,” wherein some positions may be absent
  MISC_FEATURE (431)..(435): This region may encompass “SGGQQ,” “GAGQQ,” “GQGPY,” “AGQQ” or “SQ,” wherein some positions may be absent
  MISC_FEATURE (439)..(443): This region may encompass “SGGQQ,” “GAGQQ,” “GQGPY,” “AGQQ” or “SQ,” wherein some positions may be absent
  MISC_FEATURE (447)..(451): This region may encompass “SGGQQ,” “GAGQQ,” “GQGPY,” “AGQQ” or “SQ,” wherein some positions may be absent
  MISC_FEATURE (455)..(459): This region may encompass “SGGQQ,” “GAGQQ,” “GQGPY,” “AGQQ” or “SQ,” wherein some positions may be absent
  MISC_FEATURE (463)..(467): This region may encompass “SGGQQ,” “GAGQQ,” “GQGPY,” “AGQQ” or “SQ,” wherein some positions may be absent
  MISC_FEATURE (404)..(467): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (471)..(480): This region may encompass 6-10 residues
  MISC_FEATURE (487)..(491): This region may encompass “SGGQQ,” “GAGQQ,” “GQGPY,” “AGQQ” or “SQ,” wherein some positions may be absent
  MISC_FEATURE (495)..(499): This region may encompass “SGGQQ,” “GAGQQ,” “GQGPY,” “AGQQ” or “SQ,” wherein some positions may be absent
  MISC_FEATURE (503)..(507): This region may encompass “SGGQQ,” “GAGQQ,” “GQGPY,” “AGQQ” or “SQ,” wherein some positions may be absent
  MISC_FEATURE (511)..(515): This region may encompass “SGGQQ,” “GAGQQ,” “GQGPY,” “AGQQ” or “SQ,” wherein some positions may be absent
  MISC_FEATURE (519)..(523): This region may encompass “SGGQQ,” “GAGQQ,” “GQGPY,” “AGQQ” or “SQ,” wherein some positions may be absent
  MISC_FEATURE (527)..(531): This region may encompass “SGGQQ,” “GAGQQ,” “GQGPY,” “AGQQ” or “SQ,” wherein some positions may be absent
  MISC_FEATURE (535)..(539): This region may encompass “SGGQQ,” “GAGQQ,” “GQGPY,” “AGQQ” or “SQ,” wherein some positions may be absent
  MISC_FEATURE (543)..(547): This region may encompass “SGGQQ,” “GAGQQ,” “GQGPY,” “AGQQ” or “SQ,” wherein some positions may be absent
  MISC_FEATURE (484)..(547): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (551)..(560): This region may encompass 6-10 residues
  MISC_FEATURE (567)..(571): This region may encompass “SGGQQ,” “GAGQQ,” “GQGPY,” “AGQQ” or “SQ,” wherein some positions may be absent
  MISC_FEATURE (575)..(579): This region may encompass “SGGQQ,” “GAGQQ,” “GQGPY,” “AGQQ” or “SQ,” wherein some positions may be absent
  MISC_FEATURE (583)..(587): This region may encompass “SGGQQ,” “GAGQQ,” “GQGPY,” “AGQQ” or “SQ,” wherein some positions may be absent
  MISC_FEATURE (591)..(595): This region may encompass “SGGQQ,” “GAGQQ,” “GQGPY,” “AGQQ” or “SQ,” wherein some positions may be absent
  MISC_FEATURE (599)..(603): This region may encompass “SGGQQ,” “GAGQQ,” “GQGPY,” “AGQQ” or “SQ,” wherein some positions may be absent
  MISC_FEATURE (607)..(611): This region may encompass “SGGQQ,” “GAGQQ,” “GQGPY,” “AGQQ” or “SQ,” wherein some positions may be absent
  MISC_FEATURE (615)..(619): This region may encompass “SGGQQ,” “GAGQQ,” “GQGPY,” “AGQQ” or “SQ,” wherein some positions may be absent
  MISC_FEATURE (623)..(627): This region may encompass “SGGQQ,” “GAGQQ,” “GQGPY,” “AGQQ” or “SQ,” wherein some positions may be absent
  MISC_FEATURE (564)..(627): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (631)..(640): This region may encompass 6-10 residues
  MISC_FEATURE (647)..(651): This region may encompass “SGGQQ,” “GAGQQ,” “GQGPY,” “AGQQ” or “SQ,” wherein some positions may be absent
  MISC_FEATURE (655)..(659): This region may encompass “SGGQQ,” “GAGQQ,” “GQGPY,” “AGQQ” or “SQ,” wherein some positions may be absent
  MISC_FEATURE (663)..(667): This region may encompass “SGGQQ,” “GAGQQ,” “GQGPY,” “AGQQ” or “SQ,” wherein some positions may be absent
  MISC_FEATURE (671)..(675): This region may encompass “SGGQQ,” “GAGQQ,” “GQGPY,” “AGQQ” or “SQ,” wherein some positions may be absent
  MISC_FEATURE (679)..(683): This region may encompass “SGGQQ,” “GAGQQ,” “GQGPY,” “AGQQ” or “SQ,” wherein some positions may be absent
  MISC_FEATURE (687)..(691): This region may encompass “SGGQQ,” “GAGQQ,” “GQGPY,” “AGQQ” or “SQ,” wherein some positions may be absent
  MISC_FEATURE (695)..(699): This region may encompass “SGGQQ,” “GAGQQ,” “GQGPY,” “AGQQ” or “SQ,” wherein some positions may be absent
  MISC_FEATURE (703)..(707): This region may encompass “SGGQQ,” “GAGQQ,” “GQGPY,” “AGQQ” or “SQ,” wherein some positions may be absent
  MISC_FEATURE (644)..(707): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (711)..(720): This region may encompass 6-10 residues
  MISC_FEATURE (727)..(731): This region may encompass “SGGQQ,” “GAGQQ,” “GQGPY,” “AGQQ” or “SQ,” wherein some positions may be absent
  MISC_FEATURE (735)..(739): This region may encompass “SGGQQ,” “GAGQQ,” “GQGPY,” “AGQQ” or “SQ,” wherein some positions may be absent
  MISC_FEATURE (743)..(747): This region may encompass “SGGQQ,” “GAGQQ,” “GQGPY,” “AGQQ” or “SQ,” wherein some positions may be absent
  MISC_FEATURE (751)..(755): This region may encompass “SGGQQ,” “GAGQQ,” “GQGPY,” “AGQQ” or “SQ,” wherein some positions may be absent
  MISC_FEATURE (759)..(763): This region may encompass “SGGQQ,” “GAGQQ,” “GQGPY,” “AGQQ” or “SQ,” wherein some positions may be absent
  MISC_FEATURE (767)..(771): This region may encompass “SGGQQ,” “GAGQQ,” “GQGPY,” “AGQQ” or “SQ,” wherein some positions may be absent
  MISC_FEATURE (775)..(779): This region may encompass “SGGQQ,” “GAGQQ,” “GQGPY,” “AGQQ” or “SQ,” wherein some positions may be absent
  MISC_FEATURE (783)..(787): This region may encompass “SGGQQ,” “GAGQQ,” “GQGPY,” “AGQQ” or “SQ,” wherein some positions may be absent
  MISC_FEATURE (724)..(787): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (791)..(800): This region may encompass 6-10 residues
  MISC_FEATURE (807)..(811): This region may encompass “SGGQQ,” “GAGQQ,” “GQGPY,” “AGQQ” or “SQ,” wherein some positions may be absent
  MISC_FEATURE (815)..(819): This region may encompass “SGGQQ,” “GAGQQ,” “GQGPY,” “AGQQ” or “SQ,” wherein some positions may be absent
  MISC_FEATURE (823)..(827): This region may encompass “SGGQQ,” “GAGQQ,” “GQGPY,” “AGQQ” or “SQ,” wherein some positions may be absent
  MISC_FEATURE (831)..(835): This region may encompass “SGGQQ,” “GAGQQ,” “GQGPY,” “AGQQ” or “SQ,” wherein some positions may be absent
  MISC_FEATURE (839)..(843): This region may encompass “SGGQQ,” “GAGQQ,” “GQGPY,” “AGQQ” or “SQ,” wherein some positions may be absent
  MISC_FEATURE (847)..(851): This region may encompass “SGGQQ,” “GAGQQ,” “GQGPY,” “AGQQ” or “SQ,” wherein some positions may be absent
  MISC_FEATURE (855)..(859): This region may encompass “SGGQQ,” “GAGQQ,” “GQGPY,” “AGQQ” or “SQ,” wherein some positions may be absent
  MISC_FEATURE (863)..(867): This region may encompass “SGGQQ,” “GAGQQ,” “GQGPY,” “AGQQ” or “SQ,” wherein some positions may be absent
  MISC_FEATURE (804)..(867): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (871)..(880): This region may encompass 6-10 residues
  MISC_FEATURE (887)..(891): This region may encompass “SGGQQ,” “GAGQQ,” “GQGPY,” “AGQQ” or “SQ,” wherein some positions may be absent
  MISC_FEATURE (895)..(899): This region may encompass “SGGQQ,” “GAGQQ,” “GQGPY,” “AGQQ” or “SQ,” wherein some positions may be absent
  MISC_FEATURE (903)..(907): This region may encompass “SGGQQ,” “GAGQQ,” “GQGPY,” “AGQQ” or “SQ,” wherein some positions may be absent
  MISC_FEATURE (911)..(915): This region may encompass “SGGQQ,” “GAGQQ,” “GQGPY,” “AGQQ” or “SQ,” wherein some positions may be absent
  MISC_FEATURE (919)..(923): This region may encompass “SGGQQ,” “GAGQQ,” “GQGPY,” “AGQQ” or “SQ,” wherein some positions may be absent
  MISC_FEATURE (927)..(931): This region may encompass “SGGQQ,” “GAGQQ,” “GQGPY,” “AGQQ” or “SQ,” wherein some positions may be absent
  MISC_FEATURE (935)..(939): This region may encompass “SGGQQ,” “GAGQQ,” “GQGPY,” “AGQQ” or “SQ,” wherein some positions may be absent
  MISC_FEATURE (943)..(947): This region may encompass “SGGQQ,” “GAGQQ,” “GQGPY,” “AGQQ” or “SQ,” wherein some positions may be absent
  MISC_FEATURE (884)..(947): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (951)..(960): This region may encompass 6-10 residues
  MISC_FEATURE (967)..(971): This region may encompass “SGGQQ,” “GAGQQ,” “GQGPY,” “AGQQ” or “SQ,” wherein some positions may be absent
  MISC_FEATURE (975)..(979): This region may encompass “SGGQQ,” “GAGQQ,” “GQGPY,” “AGQQ” or “SQ,” wherein some positions may be absent
  MISC_FEATURE (983)..(987): This region may encompass “SGGQQ,” “GAGQQ,” “GQGPY,” “AGQQ” or “SQ,” wherein some positions may be absent
  MISC_FEATURE (991)..(995): This region may encompass “SGGQQ,” “GAGQQ,” “GQGPY,” “AGQQ” or “SQ,” wherein some positions may be absent
  MISC_FEATURE (999)..(1003): This region may encompass “SGGQQ,” “GAGQQ,” “GQGPY,” “AGQQ” or “SQ,” wherein some positions may be absent
  MISC_FEATURE (1007)..(1011): This region may encompass “SGGQQ,” “GAGQQ,” “GQGPY,” “AGQQ” or “SQ,” wherein some positions may be absent
  MISC_FEATURE (1015)..(1019): This region may encompass “SGGQQ,” “GAGQQ,” “GQGPY,” “AGQQ” or “SQ,” wherein some positions may be absent
  MISC_FEATURE (1023)..(1027): This region may encompass “SGGQQ,” “GAGQQ,” “GQGPY,” “AGQQ” or “SQ,” wherein some positions may be absent
  MISC_FEATURE (964)..(1027): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1031)..(1040): This region may encompass 6-10 residues
  MISC_FEATURE (1047)..(1051): This region may encompass “SGGQQ,” “GAGQQ,” “GQGPY,” “AGQQ” or “SQ,” wherein some positions may be absent
  MISC_FEATURE (1055)..(1059): This region may encompass “SGGQQ,” “GAGQQ,” “GQGPY,” “AGQQ” or “SQ,” wherein some positions may be absent
  MISC_FEATURE (1063)..(1067): This region may encompass “SGGQQ,” “GAGQQ,” “GQGPY,” “AGQQ” or “SQ,” wherein some positions may be absent
  MISC_FEATURE (1071)..(1075): This region may encompass “SGGQQ,” “GAGQQ,” “GQGPY,” “AGQQ” or “SQ,” wherein some positions may be absent
  MISC_FEATURE (1079)..(1083): This region may encompass “SGGQQ,” “GAGQQ,” “GQGPY,” “AGQQ” or “SQ,” wherein some positions may be absent
  MISC_FEATURE (1087)..(1091): This region may encompass “SGGQQ,” “GAGQQ,” “GQGPY,” “AGQQ” or “SQ,” wherein some positions may be absent
  MISC_FEATURE (1095)..(1099): This region may encompass “SGGQQ,” “GAGQQ,” “GQGPY,” “AGQQ” or “SQ,” wherein some positions may be absent
  MISC_FEATURE (1103)..(1107): This region may encompass “SGGQQ,” “GAGQQ,” “GQGPY,” “AGQQ” or “SQ,” wherein some positions may be absent
  MISC_FEATURE (1044)..(1107): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1111)..(1120): This region may encompass 6-10 residues
  MISC_FEATURE (1127)..(1131): This region may encompass “SGGQQ,” “GAGQQ,” “GQGPY,” “AGQQ” or “SQ,” wherein some positions may be absent
  MISC_FEATURE (1135)..(1139): This region may encompass “SGGQQ,” “GAGQQ,” “GQGPY,” “AGQQ” or “SQ,” wherein some positions may be absent
  MISC_FEATURE (1143)..(1147): This region may encompass “SGGQQ,” “GAGQQ,” “GQGPY,” “AGQQ” or “SQ,” wherein some positions may be absent
  MISC_FEATURE (1151)..(1155): This region may encompass “SGGQQ,” “GAGQQ,” “GQGPY,” “AGQQ” or “SQ,” wherein some positions may be absent
  MISC_FEATURE (1159)..(1163): This region may encompass “SGGQQ,” “GAGQQ,” “GQGPY,” “AGQQ” or “SQ,” wherein some positions may be absent
  MISC_FEATURE (1167)..(1171): This region may encompass “SGGQQ,” “GAGQQ,” “GQGPY,” “AGQQ” or “SQ,” wherein some positions may be absent
  MISC_FEATURE (1175)..(1179): This region may encompass “SGGQQ,” “GAGQQ,” “GQGPY,” “AGQQ” or “SQ,” wherein some positions may be absent
  MISC_FEATURE (1183)..(1187): This region may encompass “SGGQQ,” “GAGQQ,” “GQGPY,” “AGQQ” or “SQ,” wherein some positions may be absent
  MISC_FEATURE (1124)..(1187): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1191)..(1200): This region may encompass 6-10 residues
  MISC_FEATURE (1207)..(1211): This region may encompass “SGGQQ,” “GAGQQ,” “GQGPY,” “AGQQ” or “SQ,” wherein some positions may be absent
  MISC_FEATURE (1215)..(1219): This region may encompass “SGGQQ,” “GAGQQ,” “GQGPY,” “AGQQ” or “SQ,” wherein some positions may be absent
  MISC_FEATURE (1223)..(1227): This region may encompass “SGGQQ,” “GAGQQ,” “GQGPY,” “AGQQ” or “SQ,” wherein some positions may be absent
  MISC_FEATURE (1231)..(1235): This region may encompass “SGGQQ,” “GAGQQ,” “GQGPY,” “AGQQ” or “SQ,” wherein some positions may be absent
  MISC_FEATURE (1239)..(1243): This region may encompass “SGGQQ,” “GAGQQ,” “GQGPY,” “AGQQ” or “SQ,” wherein some positions may be absent
  MISC_FEATURE (1247)..(1251): This region may encompass “SGGQQ,” “GAGQQ,” “GQGPY,” “AGQQ” or “SQ,” wherein some positions may be absent
  MISC_FEATURE (1255)..(1259): This region may encompass “SGGQQ,” “GAGQQ,” “GQGPY,” “AGQQ” or “SQ,” wherein some positions may be absent
  MISC_FEATURE (1263)..(1267): This region may encompass “SGGQQ,” “GAGQQ,” “GQGPY,” “AGQQ” or “SQ,” wherein some positions may be absent
  MISC_FEATURE (1204)..(1267): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1271)..(1280): This region may encompass 6-10 residues
  MISC_FEATURE (1287)..(1291): This region may encompass “SGGQQ,” “GAGQQ,” “GQGPY,” “AGQQ” or “SQ,” wherein some positions may be absent
  MISC_FEATURE (1295)..(1299): This region may encompass “SGGQQ,” “GAGQQ,” “GQGPY,” “AGQQ” or “SQ,” wherein some positions may be absent
  MISC_FEATURE (1303)..(1307): This region may encompass “SGGQQ,” “GAGQQ,” “GQGPY,” “AGQQ” or “SQ,” wherein some positions may be absent
  MISC_FEATURE (1311)..(1315): This region may encompass “SGGQQ,” “GAGQQ,” “GQGPY,” “AGQQ” or “SQ,” wherein some positions may be absent
  MISC_FEATURE (1319)..(1323): This region may encompass “SGGQQ,” “GAGQQ,” “GQGPY,” “AGQQ” or “SQ,” wherein some positions may be absent
  MISC_FEATURE (1327)..(1331): This region may encompass “SGGQQ,” “GAGQQ,” “GQGPY,” “AGQQ” or “SQ,” wherein some positions may be absent
  MISC_FEATURE (1335)..(1339): This region may encompass “SGGQQ,” “GAGQQ,” “GQGPY,” “AGQQ” or “SQ,” wherein some positions may be absent
  MISC_FEATURE (1343)..(1347): This region may encompass “SGGQQ,” “GAGQQ,” “GQGPY,” “AGQQ” or “SQ,” wherein some positions may be absent
  MISC_FEATURE (1284)..(1347): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1351)..(1360): This region may encompass 6-10 residues
  MISC_FEATURE (1367)..(1371): This region may encompass “SGGQQ,” “GAGQQ,” “GQGPY,” “AGQQ” or “SQ,” wherein some positions may be absent
  MISC_FEATURE (1375)..(1379): This region may encompass “SGGQQ,” “GAGQQ,” “GQGPY,” “AGQQ” or “SQ,” wherein some positions may be absent
  MISC_FEATURE (1383)..(1387): This region may encompass “SGGQQ,” “GAGQQ,” “GQGPY,” “AGQQ” or “SQ,” wherein some positions may be absent
  MISC_FEATURE (1391)..(1395): This region may encompass “SGGQQ,” “GAGQQ,” “GQGPY,” “AGQQ” or “SQ,” wherein some positions may be absent
  MISC_FEATURE (1399)..(1403): This region may encompass “SGGQQ,” “GAGQQ,” “GQGPY,” “AGQQ” or “SQ,” wherein some positions may be absent
  MISC_FEATURE (1407)..(1411): This region may encompass “SGGQQ,” “GAGQQ,” “GQGPY,” “AGQQ” or “SQ,” wherein some positions may be absent
  MISC_FEATURE (1415)..(1419): This region may encompass “SGGQQ,” “GAGQQ,” “GQGPY,” “AGQQ” or “SQ,” wherein some positions may be absent
  MISC_FEATURE (1423)..(1427): This region may encompass “SGGQQ,” “GAGQQ,” “GQGPY,” “AGQQ” or “SQ,” wherein some positions may be absent
  MISC_FEATURE (1364)..(1427): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1431)..(1440): This region may encompass 6-10 residues
  MISC_FEATURE (1447)..(1451): This region may encompass “SGGQQ,” “GAGQQ,” “GQGPY,” “AGQQ” or “SQ,” wherein some positions may be absent
  MISC_FEATURE (1455)..(1459): This region may encompass “SGGQQ,” “GAGQQ,” “GQGPY,” “AGQQ” or “SQ,” wherein some positions may be absent
  MISC_FEATURE (1463)..(1467): This region may encompass “SGGQQ,” “GAGQQ,” “GQGPY,” “AGQQ” or “SQ,” wherein some positions may be absent
  MISC_FEATURE (1471)..(1475): This region may encompass “SGGQQ,” “GAGQQ,” “GQGPY,” “AGQQ” or “SQ,” wherein some positions may be absent
  MISC_FEATURE (1479)..(1483): This region may encompass “SGGQQ,” “GAGQQ,” “GQGPY,” “AGQQ” or “SQ,” wherein some positions may be absent
  MISC_FEATURE (1487)..(1491): This region may encompass “SGGQQ,” “GAGQQ,” “GQGPY,” “AGQQ” or “SQ,” wherein some positions may be absent
  MISC_FEATURE (1495)..(1499): This region may encompass “SGGQQ,” “GAGQQ,” “GQGPY,” “AGQQ” or “SQ,” wherein some positions may be absent
  MISC_FEATURE (1503)..(1507): This region may encompass “SGGQQ,” “GAGQQ,” “GQGPY,” “AGQQ” or “SQ,” wherein some positions may be absent
  MISC_FEATURE (1444)..(1507): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1511)..(1520): This region may encompass 6-10 residues
  MISC_FEATURE (1527)..(1531): This region may encompass “SGGQQ,” “GAGQQ,” “GQGPY,” “AGQQ” or “SQ,” wherein some positions may be absent
  MISC_FEATURE (1535)..(1539): This region may encompass “SGGQQ,” “GAGQQ,” “GQGPY,” “AGQQ” or “SQ,” wherein some positions may be absent
  MISC_FEATURE (1543)..(1547): This region may encompass “SGGQQ,” “GAGQQ,” “GQGPY,” “AGQQ” or “SQ,” wherein some positions may be absent
  MISC_FEATURE (1551)..(1555): This region may encompass “SGGQQ,” “GAGQQ,” “GQGPY,” “AGQQ” or “SQ,” wherein some positions may be absent
  MISC_FEATURE (1559)..(1563): This region may encompass “SGGQQ,” “GAGQQ,” “GQGPY,” “AGQQ” or “SQ,” wherein some positions may be absent
  MISC_FEATURE (1567)..(1571): This region may encompass “SGGQQ,” “GAGQQ,” “GQGPY,” “AGQQ” or “SQ,” wherein some positions may be absent
  MISC_FEATURE (1575)..(1579): This region may encompass “SGGQQ,” “GAGQQ,” “GQGPY,” “AGQQ” or “SQ,” wherein some positions may be absent
  MISC_FEATURE (1583)..(1587): This region may encompass “SGGQQ,” “GAGQQ,” “GQGPY,” “AGQQ” or “SQ,” wherein some positions may be absent
  MISC_FEATURE (1524)..(1587): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1591)..(1600): This region may encompass 6-10 residues
  MISC_FEATURE (1)..(1600): This sequence may encompass 2-20 “GGY-[GPG-X1]n1-GPS-(A)n2” repeating units, wherein X1 is “SGGQQ,” “GAGQQ,” “GQGPY,” “AGQQ” or “SQ,” n1 is 4-8 and n2 is 6-10 and some positions may be absent
GGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAA

SEQ ID NO: 4 - protein, 5 aa, Artificial Sequence
  Description of Artificial Sequence Synthetic peptide
SGGQQ

SEQ ID NO: 5 - protein, 5 aa, Artificial Sequence
  Description of Artificial Sequence Synthetic peptide
GAGQQ

SEQ ID NO: 6 - protein, 5 aa, Artificial Sequence
  Description of Artificial Sequence Synthetic peptide
GQGPY

SEQ ID NO: 7 - protein, 4 aa, Artificial Sequence
  Description of Artificial Sequence Synthetic peptide
AGQQ

(19) United States
(12) Patent Application Publication
(10) Pub. No.: US 2018/0282937 A1
(43) Pub. Date: Oct. 4, 2018

(54) SUPERCONTRACTING FIBER TEXTILES

(71) Applicant: Bolt Threads, Inc., Emeryville, CA (US)

(72) Inventors: Jamie McAusland Bainbridge, Portland, OR (US); Lindsay Wray, Benicia, CA (US); Mitchell Heinrich, Oakland, CA (US); Joshua Kittleson, Pleasant Hill, CA (US); David N. Breslauer, San Francisco, CA (US)

(21) Appl. No.: 15/920,306

(22) Filed: Mar. 13, 2018

Related U.S. Application Data
(63) Continuation of application No. PCT/US2018/021818, filed on Mar. 9, 2018.
(60) Provisional application No. 62/470,151, filed on Mar. 10, 2017.

(51) Int. Cl. D06M 13/144; C07K 14/435; D06B 3/10; D02G 3/02; D02G 3/38; D04B 1/14

(52) U.S. Cl. CPC D06M 13/144; C07K 14/43518; D06B 3/10; D02G 3/02; D06M 2101/12; D04B 1/14; D10B 2401/061; D10B 2211/04; D02G 3/38

(57) ABSTRACT

The present disclosure relates generally to pre-treating textiles and methods of preparing textiles in a pre-treated state. Specifically, the present disclosure relates to pre-treating textiles comprising recombinant protein fibers.

### CROSS-REFERENCE TO RELATED APPLICATIONS

This application is a bypass continuation of and claims priority to co-pending International Application No. PCT/US2018/021818, filed Mar. 9, 2018, which claims the benefit of U.S. Provisional Patent Application No. 62/470,151, filed Mar. 10, 2017, the disclosure of which is incorporated herein by reference.

### FIELD OF THE INVENTION

The present disclosure relates generally to pre-treating textiles and methods of preparing textiles in a pre-treated state. Specifically, the present disclosure relates to pre-treating textiles comprising recombinant protein fibers.

### BACKGROUND

Most types of natural spider silk are known to shrink in length and expand in width when exposed to moisture. Since the reduction in length is large (up to approximately 50%) spider silk is considered to supercontract upon exposure to moisture. This supercontraction is not induced by heat, but rather the interaction of the water molecules with the protein structure of the fiber (Boutry, C., & T. A. Blackledge. 2010. Journal of Experimental Biology 213:3505-3514).

There are reports of recombinant protein fibers derived from spider silk also displaying supercontraction (see, e.g., US Publication No. 2011/0297904, “Humidity responsive materials and systems and methods using humidity responsive materials” incorporated by reference herein in its entirety). Supercontraction is a problem that needs to be solved in order to utilize recombinant protein fibers derived from spider silk in textiles and apparel products, because the length reduction drastically changes the shape and properties of textiles and apparel products when exposed to water. Some methods have been described to eliminate the supercontraction in recombinant protein fibers derived from spider silk by modifying the protein structure of the fiber (see, e.g., U.S. Pat. No. 9,131,671, “Methods, compositions and systems for production of recombinant spider silk polypeptides” incorporated by reference herein in its entirety). However, this process can be time consuming, provide undesirable results, and may not be amenable to all proteins or all textiles.

What is needed, therefore, are novel compositions and methods to solve the problem of creating useful textiles and apparel products that contain recombinant protein fibers derived from spider silk that supercontract upon exposure to water. Desirable compositions and methods are low cost, and have a high predictability and yield.

### SUMMARY

According to some embodiments, provided herein is a method of making a recombinant protein textile in a greige state before pre-treatment, comprising providing a yarn comprising a plurality of recombinant protein fibers, wherein said recombinant protein fibers supercontract upon exposure to water; and generating a textile from said yarn, wherein said textile has a structure suitable for generating a pre-treated textile product with desirable properties after pre-treatment of said textile to induce supercontraction of said plurality of recombinant protein fibers.

In some embodiments, the textile is a garment. In some embodiments, the garment is fully assembled prior to pre-treatment. In some embodiments, the textile is underconstructed.

In some embodiments, the textile in the greige state comprises a linear stitch density of 15-30 stitches per inch. In some embodiments, the textile comprises a linear stitch density of 15-30 stitches per inch in two perpendicular directions along a plane of the textile.

In some embodiments, the textile comprises a knitted yarn structure. In some embodiments, the textile comprises a weaved yarn structure. In some embodiments, the weave has a stitch density in the course and/or wale direction of 15-30 stitches per inch.

In some embodiments, the pre-treatment comprises exposure of said textile to water. In some embodiments, the textile is constructed such that different sections of the textile will shrink in a differential manner, thereby generating an induced shape or texture upon supercontraction. In some embodiments, the textile is a garment in the greige state. In some embodiments said textile comprises a linear stitch density of 20-60 stitches per inch after the pre-treatment. In some embodiments, the textile comprises a linear stitch density of 20-60 stitches per inch in two perpendicular directions along a plane of the textile after the pre-treatment.

In some embodiments, the recombinant protein fibers comprise a recombinant silk protein. In some embodiments, the recombinant silk protein comprises repeat units, wherein each repeat unit has at least 95% sequence identity to a sequence that comprises from 2 to 20 quasi-repeat units, each quasi-repeat unit having a composition comprising [GGY-{GPG-X1]n1-GPS-(A)n2], wherein for each quasi-repeat unit: X1 is independently selected from the group consisting of SGGQQ, GAGQQ, GQGPY, AGQQ, and SQ; and n1 is from 4 to 8, and n2 is from 6 to 10. In some embodiments, the recombinant silk protein comprises 2 or more concatenated repeats of SEQ ID NO: 1 or circularly permuted variants thereof.

Also provided herein is a device comprising special technical means and means adapted to execute the steps of the method of making a recombinant protein textile in a greige state before pre-treatment. Also provided herein is a computer program product comprising instructions to cause the device to carry out the steps of the method of making a recombinant protein textile in a greige state before pre-treatment. Also provided herein is a computer-readable medium having stored thereon the computer program for performing the steps of the method of making a recombinant protein textile in a greige state before pre-treatment on the device.

Also provided herein is a method of pre-treating a textile, comprising providing a textile comprising yarn comprising a plurality of recombinant protein fibers, wherein said textile is in a greige state; and exposing said textile to a solution comprising water or alcohol, wherein said exposure shrinks the textile, thereby providing a pre-treated textile.

In some embodiments, the method of pre-treating the textile further comprises placing said textile in a mold or on a 3 dimensional structure to constrain the shrinking of said textile during said pre-treatment, thereby generating a desired final textile shape.

In some embodiments, exposing said textile to said solution comprises submerging, dampening, moisturizing, steaming, or exposing said textile to humid conditions. In some embodiments, the textile is exposed to said solution uniformly.

In some embodiments, the exposure of said textile to said solution shrinks the area of said textile by more than 20%, or more than 25%, or more than 30%, or more than 35%, or more than 40%, or more than 45%, or from 20% to 60%, or from 20% to 50%, or from 20% to 40%, or from 20% to 30%, or from 30% to 60%, or from 30% to 50%, or from 30% to 40%, or from 40% to 60%, or from 40% to 50%. In some embodiments, the exposure of said textile to said solution shrinks by different amounts in the width and length, wherein the amount of shrinkage in the width, or the length, is from 5% to 50%, or from 10% to 50%, or from 15% to 50%, or from 20% to 50%, or from 25% to 50%, or from 5% to 40%, or from 10% to 40%, or from 15% to 40%, or from 20% to 40%, or from 25% to 40%. In some embodiments, the ratio of the amount of shrinkage in the width to the amount of shrinkage in the length is from 0 to 2, or from 0.5 to 2, or from 1 to 2, or from 1.5 to 2, or from 0 to 1.5, or from 0.5 to 1.5, or from 1 to 1.5, or from 0 to 1, or from 0.5 to 1. In some embodiments, the pre-treatment shrinks the area of said textile by from 10% to 75%, or from 20% to 75%, or from 30% to 75%, or from 40% to 75%, or from 50% to 75%, or from 10% to 60%, or from 20% to 60%, or from 30% to 60%, or from 40% to 60%, or from 50% to 60%.

In some embodiments, tension is applied to said textile during said exposure of said textile to said solution. In some embodiments, the tension is from 1 to 100 MPa, or from 20 to 80 MPa, or from 30 to 70 MPa, or from 40 to 60 MPa, or greater than 60 MPa, or greater than 100 MPa. In some embodiments, the textile is exposed to water at a temperature at a temperature below 50° C., or below 45° C., or below 40° C., or below 35° C., or below 30° C., or is exposed to water at a temperature of from 15° C. to 50° C., or from 15° C. to 45° C., or from 15° C. to 40° C., or from 15° C. to 35° C., or from 15° C. to 30° C., or from 15° C. to 25° C., or at approximately 20° C.

In some embodiments, the solution is greater than 99%, greater than 90%, greater than 80%, greater than70%, or greater than 60% water or alcohol, or a mixture thereof. In some embodiments, the alcohol is selected from the group consisting of: a monohydric alcohol, a polyhydric alcohol, an unsaturated aliphatic alcohol, and an alicyclic alcohol. In some embodiments, the alcohol is selected from the group consisting of: methanol, ethanol, propanol, butanol, and pentanol.

In some embodiments, the solution comprises an additive. In some embodiments, the additive is selected from the group consisting of: a lubricating additive, a cohesive additive, a surfactant, or an additive to facilitate supercontraction.

In some embodiments, the method of pre-treating the textile further comprises drying said textile after said exposure of said textile to water. In some embodiments, the drying is performed at a temperature from 15° C. to 50° C., or from 15° C. to 45° C., or from 15° C. to 40° C., or from 15° C. to 35° C., or from 15° C. to 30° C., or from 15° C. to 25° C., or at approximately 20° C. In some embodiments, tension is applied to said textile during drying. In some embodiments, the tension is from 1 to 100 MPa, or from 20 to 80 MPa, or from 30 to 70 MPa, or from 40 to 60 MPa, or greater than 60 MPa, or greater than 100 MPa.

In some embodiments, the pre-treated textile is resistant to shrinkage or machine washable as compared to before said pre-treatment. In some embodiments, the pre-treated textile has a shrinkage along one or more axes of less than 15%, or less than 10%, or less than 5%, or from 0% to 15%, or from 0% to 10%, or from 0% to 5% upon subsequent exposure to water. In some embodiments, the pre-treated textile has a shrinkage in area of less than 15%, or less than 10%, or less than 5%, or from 0% to 15%, or from 0% to 10%, or from 0% to 5% upon subsequent exposure to water. In some embodiments, the resistance to shrinkage is as tested by AATCC TM135-2015 Dimensional Changes of Fabrics after Home Laundering, AATCC TM96-2012, Dimensional Changes in Commercial Laundering of Woven and Knitted Fabrics Except Wool, AATCC TM187-2013, Dimensional Changes of Fabrics: Accelerated, or AATCC TM150-2012, Dimensional Changes of Garments after Home Laundering.

In some embodiments, the textile is a garment. In some embodiments, the pre-treatment is performed while wearing said garment. In some embodiments, the textile is a knitted textile, a woven textile, or a non-woven textile. In some embodiments, the textile is a fabric. In some embodiments, the fabric is a knitted fabric or a woven fabric. In some embodiments, the textile is selected from the group consisting of a needle punched textile, a spunlace textile, a wet-laid textile, a dry-laid textile, a melt-blown textile, and 3-D printed non-woven textile. In some embodiments, the textile is selected from the group consisting of a circular-knitted textile, flat-knitted textile, a push-pull textile, or a warp-knitted textile. In some embodiments, the textile comprises a microknit or a microweave after said pre-treatment.

In some embodiments, the yarn comprises an outer sheath comprising the recombinant protein fiber, wherein the outer sheath comprises a greater twist as compared to a twist in a center core of the filament yarn; and wherein the mean dernier of the recombinant protein fiber is less than 5. In some embodiments, the yarn is a blended yarn comprising recombinant protein fibers and fibers selected from the group consisting of cotton, wool, merino, mohair, polyamide, linen, acrylic, polyester, spandex, and combinations thereof.

In some embodiments, the recombinant protein fibers comprise a recombinant silk protein. In some embodiments, the recombinant silk protein comprises repeat units, wherein each repeat unit has at least 95% sequence identity to a sequence that comprises from 2 to 20 quasi-repeat units, each quasi-repeat unit having a composition comprising [GGY-{GPG-X1]n1-GPS-(A)n2], wherein for each quasi-repeat unit: X1 is independently selected from the group consisting of SGGQQ, GAGQQ, GQGPY, AGQQ, and SQ; and n1 is from 4 to 8, and n2 is from 6 to 10. In some embodiments, the recombinant silk protein comprises 2 or more concatenated repeats of SEQ ID NO: 1 or circularly permuted variants thereof.

Also provided herein is a textile comprising yarn comprising a plurality of recombinant protein fibers, wherein said textile is in a greige state, and wherein said textile supercontracts upon exposure to water. In some embodiments, the textile is a garment. In some embodiments, the textile is fully assembled prior to pre-treatment.

In some embodiments, the textile is underconstructed. In some embodiments, the textile comprises a linear stitch density of 15-30 stitches per inch. In some embodiments, the textile comprises a linear stitch density of 15-30 stitches per inch in two perpendicular directions along a plane of the textile.

In some embodiments, the textile comprises a knitted yarn structure or a weaved yarn structure. In some embodiments, the textile is constructed such that different sections of the textile will shrink in a differential manner, thereby generating an induced shape or texture upon supercontraction.

In some embodiments, the supercontraction shrinks the area of said textile by more than 20%, or more than 25%, or more than 30%, or more than 35%, or more than 40%, or more than 45%, or from 20% to 60%, or from 20% to 50%, or from 20% to 40%, or from 20% to 30%, or from 30% to 60%, or from 30% to 50%, or from 30% to 40%, or from 40% to 60%, or from 40% to 50%.

In some embodiments, the plurality of recombinant protein fibers comprise a recombinant silk protein. In some embodiments, the recombinant silk protein comprises repeat units, wherein each repeat unit has at least 95% sequence identity to a sequence that comprises from 2 to 20 quasi-repeat units, each quasi-repeat unit having a composition comprising [GGY-{GPG-X1]n1-GPS-(A)n2], wherein for each quasi-repeat unit: X1 is independently selected from the group consisting of SGGQQ, GAGQQ, GQGPY, AGQQ, and SQ; and n1 is from 4 to 8, and n2 is from 6 to 10. In some embodiments, the recombinant silk protein comprises 2 or more concatenated repeats of SEQ ID NO: 1 or circularly permuted variants thereof.

Also provided herein is a textile comprising yarn comprising a plurality of recombinant protein fibers, wherein said textile has been pre-treated. In some embodiments, the textile is a garment. In some embodiments, the textile comprises a linear stitch density of 20-60 stitches per inch. In some embodiments, the textile comprises a linear stitch density of 20-60 stitches per inch in two perpendicular directions along a plane of the textile. In some embodiments, the supercontraction shrinks said plurality of recombinant fibers by 20% or more.

In some embodiments, the pre-treated textile has a shrinkage along one or more axes of less than 15%, or less than 10%, or less than 5%, or from 0% to 15%, or from 0% to 10%, or from 0% to 5% upon subsequent exposure to water. In some embodiments, the textile has a shrinkage in area of less than 15%, or less than 10%, or less than 5%, or from 0% to 15%, or from 0% to 10%, or from 0% to 5% upon subsequent exposure to water.

In some embodiments, the plurality of recombinant protein fibers comprise a recombinant silk protein. In some embodiments, the recombinant silk protein comprises repeat units, wherein each repeat unit has at least 95% sequence identity to a sequence that comprises from 2 to 20 quasi-repeat units, each quasi-repeat unit having a composition comprising [GGY-{GPG-X1]n1-GPS-(A)n2], wherein for each quasi-repeat unit: X1 is independently selected from the group consisting of SGGQQ, GAGQQ, GQGPY, AGQQ, and SQ; and n1 is from 4 to 8, and n2 is from 6 to 10. In some embodiments, the recombinant silk protein comprises 2 or more concatenated repeats of SEQ ID NO: 1 or circularly permuted variants thereof.

### BRIEF DESCRIPTION OF THE DRAWINGS

FIGS. 1A and 1B schematically illustrate two different knitted patterns, a reverse jersey (FIG. 1A) and a decorative pattern (FIG. 1B), for a textile in the greige state.

FIG. 2 is a flow chart depicting a method to pre-treat recombinant protein textiles (RPTs).

FIG. 3 is a flow chart depicting a method to pre-treat garments containing recombinant protein fibers (RPFs).

FIG. 4 schematically illustrates a decorative knitted pattern in a greige state.

FIG. 5 shows photographs of an RPT with a decorative knitted pattern before and after pre-treatment.

FIG. 6 shows photographs of an RPT with a reverse jersey knitted pattern after pre-treatment.

FIG. 7 shows photographs of a tow of RPFs before and after pre-treatment, illustrating supercontraction after pre-treatment.

FIG. 8 shows photographs of a knitted RPT before and after pre-treatment, where the RPT shrinks by different amounts in the course and wale directions.

FIG. 9 shows scanning electron microscope (SEM) images of a knitted RPT before and after pre-treatment, illustrating the changes to the stitching pattern and density of the RPT due to pre-treatment, according to an embodiment.

FIGS. 10A, 10B, 10C, 10D, and 10E show photographs of jersey knitted RPTs before and after pre-treatment.

The figures depict various embodiments of the present disclosure for purposes of illustration only. One skilled in the art will readily recognize from the following discussion that alternative embodiments of the structures and methods illustrated herein may be employed without departing from the principles described herein.

### ABBREVIATIONS AND DEFINITIONS

Unless defined otherwise, all technical and scientific terms used herein have the same meaning as is commonly understood by one of ordinary skill in the art to which this disclosure pertains.

The terms “a” and “an” and “the” and similar referents as used herein refer to both the singular and the plural, unless otherwise indicated herein or clearly contradicted by context.

The term “about,” “approximately,” or “similar to” means within an acceptable error range for the particular value as determined by one of ordinary skill in the art, which can depend in part on how the value is measured or determined, or on the limitations of the measurement system. It should be understood that all ranges and quantities described below are approximations and are not intended to limit the invention. Where ranges and numbers are used these can be approximate to include statistical ranges or measurement errors or variation. In some embodiments, for instance, measurements could be plus or minus 10%.

Amino acids can be referred to by their single-letter codes or by their three-letter codes. The single-letter codes, amino acid names, and three-letter codes are as follows: G—Glycine (Gly), P—Proline (Pro), A—Alanine (Ala), V—Valine (Val), L—Leucine (Leu), I—Isoleucine (Ile), M—Methionine (Met), C—Cysteine (Cys), F—Phenylalanine (Phe), Y—Tyrosine (Tyr), W—Tryptophan (Trp), H—Histidine (His), K—Lysine (Lys), R—Arginine (Arg), Q—Glutamine (Gln), N—Asparagine (Asn), E—Glutamic Acid (Glu), D—Aspartic Acid (Asp), S—Serine (Ser), T—Threonine (Thr).

The terms “including,” “includes,” “having,” “has,” “with,” or variants thereof are intended to be inclusive in a manner similar to the term “comprising”.

The term “microbe” as used herein refers to a microorganism, and refers to a unicellular organism. As used herein, the term includes all bacteria, all archaea, unicellular protista, unicellular animals, unicellular plants, unicellular fungi, unicellular algae, all protozoa, and all chromista.

The term “native” as used herein refers to compositions found in nature in their natural, unmodified state.

The terms “optional” or “optionally” mean that the feature or structure may or may not be present, or that an event or circumstance may or may not occur, and that the description includes instances where a particular feature or structure is present and instances where the feature or structure is absent, or instances where the event or circumstance occurs and instances where the event or circumstance does not occur.

The term “recombinant” refers to a biomolecule, e.g., a gene or protein, that has been removed from its naturally occurring environment, is not associated with all or a portion of a polynucleotide in which the gene is found in nature, is operatively linked to a polynucleotide which it is not linked to in nature, or does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids. A recombinant protein can refer to a non-native polypeptide that is produced in a host cell, or to a polypeptide synthesized from a recombinant nucleic acid. A recombinant nucleic acid refers to a nucleic acid that is removed from its naturally occurring environment, or a nucleic acid that is not associated with all or a portion of a nucleic acid abutting or proximal to the nucleic acid when it is found in nature, or a nucleic acid that is operatively linked to a nucleic acid that it is not linked to in nature, or a nucleic acid that does not occur in nature, or a nucleic acid that contains a modification that is not found in that nucleic acid in nature (e.g., insertion, deletion, or point mutation introduced artificially, e.g., by human intervention), or a nucleic acid that is integrated into a chromosome at a heterologous site. The term includes cloned DNA isolates and nucleic acids that comprise a chemically-synthesized nucleotide analog.

The term “recombinant host cell” as used herein refers to a host cell that comprises a recombinant nucleic acid.

The term “repeat” as used herein, in reference to an amino acid or nucleic acid sequence, refers to a sub-sequence that is present more than once in a polynucleotide or polypeptide (e.g., a concatenated sequence). A polynucleotide or polypeptide can have a direct repetition of the repeat sequence without any intervening sequence, or can have non-consecutive repetition of the repeat sequence with intervening sequences. The term “quasi-repeat” as used herein, in reference to amino acid or nucleic acid sequences, is a sub-sequence that is inexactly repeated (i.e., wherein some portion of the quasi-repeat subsequence is variable between quasi-repeats) across a polynucleotide or polypeptide. Repeating polypeptides and DNA molecules (or portions of polypeptides or DNA molecules) can be made up of either repeat sub-sequences (i.e., exact repeats) or quasi-repeat sub-sequences (i.e., inexact repeats).

In the present disclosure, the term “greige state” refers the state of the textile after textile construction (e.g., weaving knitting, etc.) and before pre-treatment.

In the present disclosure, the term “under-constructed” refers to a textile that has a gap size between stitches, a stitch length and/or a stitch density that is larger than the intended gap size between stitches, stitch length and/or stitch density of the final product.

In the present disclosure, the term “recombinant protein fiber” (RPF), refers to a fiber comprising recombinant proteins. Recombinant protein fibers (RPFs) are fibers that are produced from recombinant proteins. In some cases, the proteins making up the RPFs can contain concatenated repeat units and quasi-repeat units. Repeat units are defined as amino acid sequences that are repeated exactly within the polypeptide. Quasi-repeats are inexact repeats, i.e., there is some sequence variation from quasi-repeat to quasi-repeat. Each repeat can be made up of concatenated quasi-repeats.

In the present disclosure, the term “recombinant protein yarn” (RPY) refers to a yarn comprising a plurality of RPFs twisted around a common axis (or set of axes in the case of plied yarns).

In the present disclosure, the term “recombinant protein textile” (RPT) refers to textiles comprising RPYs. In some embodiments, an RPT is a knitted, woven or non-woven textile.

In the present disclosure, the term “supercontract” (or, “supercontraction”) refers to the shrinking (i.e., decreasing in length) of a recombinant protein fiber (RPF) by more than 20% when exposed to moisture, and the RPF is unconstrained. Unlike shrinking in typical fibers (e.g., cotton and wool), no heat or agitation is needed for supercontraction to occur in RPFs. In some embodiments, supercontraction occurs when RPFs are exposed to moisture at a temperature from 10° C. to 50° C., or from 15° C. to 50° C., or from 20° C. to 50° C., or from 20° C. to 40° C., or from 20° C. to 30° C., or from 20° C. to 25° C. (i.e., near room temperature). In some embodiments, supercontraction occurs when the RPF is exposed to moisture by exposing the RPF to water vapor at a relative humidity (RH) greater than 60%, or greater than 70%, or greater than 75%, or greater than 80%, or greater than 85%, or greater than 90%. In some embodiments, supercontraction occurs when the RPF is exposed to moisture by submerging the RPF in a solution, where the solution is water, or greater than 99% water, or greater than 90% water, or greater than 80% water, or greater than 70% water, or greater than 60% water, or water with some additives. In embodiments where the RPF is constrained and exposed to moisture, the RPF exerts a force from approximately 40 MPa to approximately 60 MPa (resulting from the tendency of the RPF to supercontract).

In the present disclosure, the term “under-constructed” refers to a textile that has a stitch density (i.e., number of stitches per unit length) that is less than the intended stitch density of the final product.

In the present disclosure, the term “greige state” refers the state of the textile after textile construction (e.g., weaving knitting, etc.) and before pre-treatment.

In the present disclosure, the term “filament yarns” refers to yarns that are composed of more than one fiber filaments that run the whole length of the yarn. Filament yarns can also be referred to as multi-filament yarns. The structure of a filament yarn is influenced by the amount of twist, and in some cases the fiber texturing. The properties of the filament yarn can be influenced by the structure of the yarn, fiber to fiber friction of the constituent fibers, and the properties of the constituent fibers. In some embodiments, the yarn structure and the recombinant protein fiber properties are chosen to impart various characteristics to the resulting yarns. The properties of the yarn can also be influenced by the number of fibers (i.e., filaments) in the yarn. The filament yarns in this application can be multifilament yarns. Throughout this disclosure “filament yarns” can refer to flat filament yarns, textured filament yarns, drawn filament yarns, undrawn filament yarns, or filament yarns of any structure.

In the present disclosure, the term “spun yarn” refers to a yarn that is made by twisting staple fibers together to make a cohesive yarn (or thread, or “single”). The structure of a spun yarn is influenced by the spinning methods parameters. The properties of the spun yarn are influenced by the structure of the yarn, as well as the constituent fibers.

In the present disclosure, the term “blended yarns” refer to a type of yarn comprising various fibers being blended together. In different embodiments, the recombinant protein fibers can be blended with cotton, wool, other animal fibers, polyamide, acrylic, nylon, linen, polyester, and/or combinations thereof. Recombinant protein fibers can be blended with non-recombinant protein fibers (non-RPFs), or with more than one other type of non-recombinant protein fibers. Recombinant protein fibers can also be blended with a second type of recombinant protein fiber with different properties than the first type of recombinant protein fibers. In this disclosure, blended yarns specifically refer to recombinant protein fibers (RPFs) blended with non-recombinant protein fibers or a second type of recombinant protein fibers into a yarn. Even though spandex is generally incorporated into a yarn using somewhat different methods and structures than the other blended yarns described above (e.g., a wrapped RFP/spandex yarn has spandex core wrapped with RPF in order to hide the spandex from view in the textile), a composite RPF/spandex yarn therefore is another example of a blended yarn.

The standard test method for measuring tensile properties of yarns (or multiple fibers in a tow) by the single-strand method is ASTM D2256-10. The standard test method for measuring tensile properties of single fibers is ASTM D3822-14. All fiber and yarn mechanical properties measured in this disclosure are measured using one of these standards.

“Textured” fibers or yarns are fibers or yarns that have been subjected to processes that arrange the straight filaments into crimped, coiled or looped filaments. Some examples of methods used for processing textured fibers and yarns are air jet texturing, false twist texturing, or stuffer box texturing.

The “work of rupture” of a fiber or yarn is the work done from the point of the pretension load to the point of the breaking load. The energy required to bring a fiber or yarn to the breaking load can be obtained from the area under the load-elongation curve. The units of work of rupture can therefore be cN*cm. The “toughness” of a fiber or yarn is the energy per unit mass required to rupture the fiber or yarn. The toughness is the integral of the stress-strain curve, and can be calculated by dividing the work of rupture by the mass of the sample of fiber or yarn being tested. The units of toughness can therefore be cN/tex.

Throughout this disclosure, and in the claims, when percentages of amino acids are recited, that percentage indicates a mole fraction percentage (not a weight fraction percentage).

Throughout this disclosure, and in the claims, where method steps are recited, the order in which the steps are carried out can be varied from the order in which they are described, so long as an operable method results.

Recitation of ranges of values herein are merely intended to serve as a shorthand method of referring individually to each separate value inclusively falling within the range, unless otherwise indicated herein, and each separate value is incorporated into the specification as if it were individually recited herein.

### DETAILED DESCRIPTION

The present disclosure describes recombinant protein textiles (RPTs), which comprise a plurality recombinant protein yarns (RPYs), which comprise a plurality of recombinant protein fibers (RPFs). In some embodiments, the RPTs are under-constructed in the greige state, such that when the RPT is pre-treated (e.g., by exposing to water at room temperature) the RPT shrinks and the resulting pre-treated RPT has desirable construction and properties. In some embodiments, the RPT is a knitted, woven or non-woven textile.

In some embodiments, the RPFs described herein supercontract upon exposure to moisture. In some embodiments, RPTs comprise RPFs that supercontract upon exposure to moisture, and the RPTs are under-constructed in the greige state.

The present disclosure discusses methods by which an RPT is pre-treated by exposing said textile to water, wherein said exposure shrinks the textile creating a pre-treated RPT. In some embodiments, methods for pre-treating an RPT comprise exposing said textile to water, wherein said exposure shrinks the textile creating a pre-treated RPT, and wherein all steps of the pre-treatment occur at a temperature below 50° C., or below 45° C., or below 40° C., or below 35° C., or below 30° C.

In some embodiments, methods for pre-treating an RPT comprise exposing said textile to water, wherein said exposure shrinks the textile creating a pre-treated RPT, and wherein the RPT comprises RPFs that supercontract upon exposure to moisture. In some embodiments, methods for pre-treating an RPT comprise exposing said textile to water, wherein said exposure shrinks the textile creating a pre-treated RPT, and wherein the pre-treatment occurs at a temperature below 50° C., or below 45° C., or below 40° C., or below 35° C., or below 30° C., and wherein the RPTs comprise RPFs supercontract upon exposure to moisture.

In some embodiments, the RPTs of the present disclosure comprise RPFs that exhibit supercontraction upon exposure to moisture, and shrink by more than 20%, or more than 25%, or more than 30%, or more than 35%, or more than 40%, or more than 45%, or from 20% to 60%, or from 20% to 50%, or from 20% to 40%, or from 20% to 30%, or from 30% to 60%, or from 30% to 50%, or from 30% to 40%, or from 40% to 60%, or from 40% to 50%.

PRE-TREATED RPT PROPERTIES

This section describes the properties of RPTs before and after pre-treatment. The pre-treatment methods are generally described above, and described below in more detail. One non-limiting example of a pre-treatment method is to provide a knitted RPT, submerge the knitted RPT in water at a temperature from 15° C. to 50° C. without applying tension, and dry in air at a temperature from 15° C. to 50° C.

In the present disclosure, an RPT can be a knitted textile, a woven textile, or a non-woven textile. Some examples of knitted RPTs are circular-knitted textiles, flat-knitted textiles, and warp-knitted textiles. Some examples of woven RPTs are plain weave textiles, dobby weave textiles, and jacquard weave textiles. Some examples of non-woven RPTs are needle punched textiles, spunlace textiles, wet-laid textiles, dry-laid textiles, melt-blown textiles, and 3-D printed non-woven textiles.

Knitted RPTs are made up of different patterns of different types of knitted stitches. Knitting machines can produce knitted RPTs using different types of stitches, in different patterns to produced different types of RPTs and garments. The nomenclature for the types of stitches varies by knitting machine manufacturer, but three basic types of stitches can be referred to as the knit, the tuck, and the slip. If the pattern of stitches in an RPT is varied, then the knitted RPTs produced will have different appearance, and will also have different physical and mechanical properties (e.g., drapability) before and/or after pre-treatment. In some embodiments, a knitted pattern is created when a certain set of stitches is defined and repeated, or shifted and repeated, in subsequent rows of an RPT. Many different knitted patterns are possible by combining different stitches in different repeated, or shifted and repeated, patterns.

In some embodiments, the tension of the stitches while producing an RPT on a knitting machine can vary. In some embodiments, varying the tension of the stitches within a pattern will produce RPTs with different appearance, and different physical and mechanical properties (e.g., visual appearance, or drapability) before and/or after pre-treatment.

In addition to the pattern of stitches, the gauge of the knitting machine can also vary. In some embodiments, varying the gauge of the knitting machine will produce RPTs with different appearance, and different physical and mechanical properties (e.g., drapability) before and/or after pre-treatment.

In some embodiments, RPTs with different knitted patterns, gauge and/or tension are pre-treated, and the different RPTs will react differently during pre-treatment. In some embodiments, RPTs with different knitted patterns, gauge and/or tension are pre-treated by submerging in water, and the different RPTs will shrink by different amounts during pre-treatment. In some embodiments, the knitted pattern, gauge and/or tension of the RPT will cause the RPT to shrink differently in the course and wale directions during pre-treatment.

RPTs with different knitted patterns may have different degrees of shrinkage during pre-treatment (e.g., submerging in water). Furthermore, knitted RPTs with different knitted patterns may have different ratios of shrinkage in the course and wale directions during pre-treatment (e.g., submerging in water). Not to be limited by theory, RPTs with different knitted patterns will have different average orientation angles with respect to the wale and course directions, and will have greater or fewer entanglements, as well as experience different amounts of tension (caused by the knitted stitches), all of which may affect the amount of shrinkage that occurs during pre-treatment (e.g., as the RPFs supercontract).

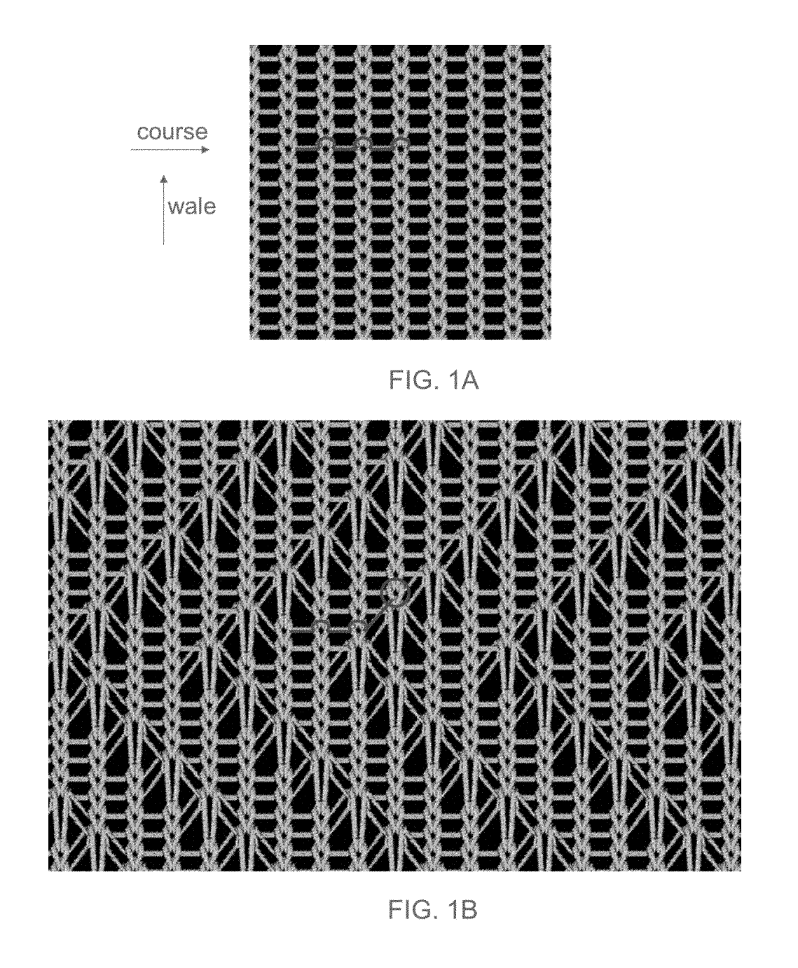

FIGS. 1A and 1B show computer-generated representations of two different knitted patterns in RPTs in embodiments. Note that FIGS. 1A and 1B are not drawn to scale, and in actual knitted RPTs the diameter of the yarn may be thicker or thinner than that shown, and the spacing between the courses and the wales may be different than what is shown in the figures. FIG. 1A shows a simple reverse jersey pattern and FIG. 1B shows a more complex pattern having a base of a reverse jersey pattern, and tuck stitches and jersey stitches in a zig-zag pattern that create a reverse herringbone. The repeat length of the pattern in FIG. 1B is 24 courses: the herringbone zig-zags proceeded in one direction for 12 courses, and then back the opposite direction for 12 courses, and then the pattern repeats. The reverse jersey knitted pattern in FIG. 1A is formed from yarns that travel in the course direction (horizontal in FIG. 1A), and then form a loop that interlocks with a length of yarn in the row above. The knitted pattern in FIG. 1B is formed from yarns that travel in the course direction (horizontal in FIG. 1A), and then change direction and travel in a diagonal direction (moving in the course and wale directions) before interlocking with a length of yarn in a more complex arrangement (depicted with an open circle in FIG. 1B). In some embodiments, RPTs with the knitted pattern shown in FIG. 1A will shrink more than RPTs with the knitted pattern shown in FIG. 1B in pre-treatment. For example, when pre-treated by exposing to water, an RPT with a knitted pattern shown in FIG. 1A would shrink more than an RPT with a knitted pattern shown in FIG. 1B. The RPYs making up an RPT with the knitted pattern shown in FIG. 1A are oriented in the course direction to a larger degree than the RPYs in an RPT with the knitted pattern shown in FIG. 1B. Not to be limited by theory, the average orientation of the RPYs in an RPT is one factor that affects the degree to which the RPT shrinks when the RPTs supercontract during pre-treatment. Furthermore, in some embodiments, the average orientation of the RPYs affects the ratio of shrinking in the course and wale directions in RPTs during pre-treatment.

In some embodiments, the RPT is a push-pull textile. In some embodiments, the RPT is a knitted or woven textiles with a push/pull construction. For example, textiles may be knit using a double knit construction either circular or warp knit where the hydrophobic RPFs or non-RPFs can be located in a layer next to the skin, and hydrophilic RPFs or non-RPFs can be located in a layer away from the skin, so that the moisture can be carried along the outside of the fiber through capillary action. Once the moisture reaches the outer hydrophilic fibers it is spread quickly across the outer surface of the fabric where it can evaporate and hence keep the wearer drier and more comfortable. In another example, textiles may be woven using a double weave construction where the hydrophobic RPFs or non-RPFs can be located in a layer next to the skin, and hydrophilic RPFs or non-RPFs yarns can be located in a layer away from the skin, so that the moisture can be carried along the outside of the fiber through capillary action.

In some embodiments, the RPT comprises a microknit or a microweave after said treatment. In some embodiments, the median or mean denier of the RPFs comprising the RPT is less than 1 denier (about 15 microns in diameter). In some embodiments, the median or mean denier of the RPFs comprising the RPT is less than 0.5 denier (about 10 microns in diameter). Microfibers are a classification of fibers having a fineness of less than 1 decitex (dtex), approximately 10 μm in diameter. H. K., Kaynak and O. Babaarslan, Woven Fabrics, Croatia: InTech, 2012. The small diameter of microfibers imparts a range of qualities and characteristics to microfiber yarns and microfiber fabrics (i.e., microknit or microweave RPTs) that are desirable to consumers. RPTs comprising microfibers are inherently more flexible (bending is inversely proportional to fiber diameter) and thus have a soft feel, low stiffness, and high drapability. Microfibers can also be formed into filament yarns having high fiber density (greater fibers per yarn cross-sectional area), giving microfiber yarns a higher strength compared to other yarns of similar dimensions. Microfibers also contribute to discrete stress relief within the yarn, resulting in anti-wrinkle RPTs. Furthermore, microfibers have high compaction efficiency within the yarn, which improves RPT waterproofness and windproofness while maintaining breathability compared to other waterproofing and windproofing techniques (such as polyvinyl coatings). The high density of fibers within microfiber RPTs results in microchannel structures between fibers, which promotes the capillary effect and imparts a wicking and quick drying characteristic. The high surface area to volume of microfiber yarns allows for brighter and sharper dyeing, and printed RPTs have clearer and sharper pattern retention as well.

In some embodiments, pre-treatment generates a pre-treated RPT (i.e., an RPT after pre-treatment) that is resistant to shrinkage or is machine washable as compared to before said pre-treatment. In some embodiments, pre-treatment generates a pre-treated RPT that has a shrinkage of less than 15%, or less than 10%, or less than 5%, or from 0% to 15%, or from 0% to 10%, or from 0% to 5%. In some embodiments, the RPT is a knitted textile and the pre-treated RPT has a shrinkage in the wale and/or course direction of less than 15%, or less than 10%, or less than 5%, or from 0% to 15%, or from 0% to 10%, or from 0% to 5%. In some embodiments, the RPT is a knitted textile and the pre-treated RPT has a shrinkage in the wale and/or course direction of different amounts. In some embodiments, the RPT is a woven textile and the pre-treated RPT has a shrinkage in the warp and/or weft direction of less than 15%, or less than 10%, or less than 5%, or from 0% to 15%, or from 0% to 10%, or from 0% to 5%. In some embodiments, the RPT is a woven textile and the pre-treated RPT has a shrinkage in the warp and/or weft direction of different amounts. Examples of test methods that can be used to determine the resistance to shrinkage are AATCC TM135-2015 Dimensional Changes of Fabrics after Home Laundering, AATCC TM96-2012, Dimensional Changes in Commercial Laundering of Woven and Knitted Fabrics Except Wool, AATCC TM187-2013, Dimensional Changes of Fabrics: Accelerated, and AATCC TM150-2012, Dimensional Changes of Garments after Home Laundering.

In some embodiments, an RPT after pre-treatment has desirable properties, such as mechanical properties, aesthetic properties, comfort properties, biological properties, colorfastness, laundering properties, moisture management properties, or other properties (e.g., those tested for by the AATCC standard test methods and evaluation procedures). In some embodiments, an RPT is under-constructed in the greige state, is pre-treated, and has desirable properties in the final state. In some embodiments, the desirable property of the final RPT is desirable drapability.

In some embodiments, RPTs are under-constructed in the greige state, such that a desired construction is achieved after shrinking in pre-treatment. In some embodiments, the knit tension in the knitting machine is calculated using the known shrinkage of the yarns in the course and wale directions. In some embodiments, the RPTs are under-constructed by adding the percent of shrinkage to the dimension of the knit program in each direction in order to produce a textile that would finish to the correct size once it underwent the pre-treating process.

In some embodiments, an RPT before or after pre-treatment has an average or median weight from 10 to 500 g/m², or from 50 to 400 g/m², or from 50 to 350 g/m², or from 150 to 250 g/m², or from 10 to 100 g/m², or from 300 to 500 g/m², or from 200 to 500 g/m², or from 100 to 400 g/m².

In some embodiments, the RPT before or after pre-treatment has an average or median stitch density from 3 stitches per inch (SPI) to 50 SPI, or from 5 SPI to 50 SPI, or from 10 SPI to 40 SPI, or from 10 SPI to 30 SPI, or from 10 SPI to 20 SPI in the course direction or from 3 SPI to 50 SPI, or from 5 SPI to 50 SPI, or from 10 SPI to 40 SPI, or from 10 SPI to 30 SPI, or from 10 SPI to 20 SPI in the wale direction before said pre-treatment, or after said pre-treatment.

In some embodiments, the RPT before or after pre-treatment has high moisture wicking properties. A standard method of measuring wicking rate is the AATCC test method 197-2011 for vertical wicking of textiles, and AATCC test method 198-2011 for horizontal wicking of textiles. In some embodiments, an RPT that is a plain weave 1/1 textile with warp density of 72 warps/cm and pick density of 40 picks/cm is tested using AATCC test method 197-2011, and has a median or mean horizontal wicking rate greater than 1 mm/s, or a median or mean horizontal wicking rate from 0.1 to 100 mm/s, or a median or mean horizontal wicking rate greater than 0.1 mm/s, or has a median or mean horizontal wicking rate greater than 0.2 mm/s, or has a median or mean horizontal wicking rate greater than 0.4 mm/s, or has a median or mean horizontal wicking rate greater than 0.6 mm/s, or has a median or mean horizontal wicking rate greater than 0.8 mm/s, or has a median or mean horizontal wicking rate greater than 2 mm/s, or has a median or mean horizontal wicking rate greater than 4 mm/s, or has a median or mean horizontal wicking rate greater than 6 mm/s, or has a median or mean horizontal wicking rate greater than 8 mm/s, or has a median or mean horizontal wicking rate greater than 10 mm/s, or has a median or mean horizontal wicking rate greater than 15 mm/s, or has a median or mean horizontal wicking rate greater than 20 mm/s, or has a median or mean horizontal wicking rate greater than 40 mm/s, or has a median or mean horizontal wicking rate greater than 60 mm/s, or has a median or mean horizontal wicking rate greater than 80 mm/s, or has a median or mean horizontal wicking rate greater than 100 mm/s. In some embodiments, an RPT that is a plain weave 1/1 textile with warp density of 72 warps/cm and pick density of 40 picks/cm is tested using AATCC test method 197-2011, and has a median or mean horizontal wicking rate from 0.1 mm/s to 1 mm/s, or has a median or mean horizontal wicking rate from 1 mm/s to 10 mm/s, or has a median or mean horizontal wicking rate from 10 mm/s to 20 mm/s, or has a median or mean horizontal wicking rate from 20 mm/s to 30 mm/s, or has a median or mean horizontal wicking rate from 30 mm/s to 40 mm/s, or has a median or mean horizontal wicking rate from 40 mm/s to 50 mm/s, or has a median or mean horizontal wicking rate from 50 mm/s to 60 mm/s, or has a median or mean horizontal wicking rate from 60 mm/s to 70 mm/s, or has a median or mean horizontal wicking rate from 70 mm/s to 80 mm/s, or has a median or mean horizontal wicking rate from 80 mm/s to 90 mm/s, or has a median or mean horizontal wicking rate from 90 mm/s to 100 mm/s. RPTs with desirable moisture wicking properties are useful in textiles and garments such as skin knits or woven fabrics where wicking of moisture away from the skin is desired, such as active wear apparel. In some embodiments, RPTs with desirable moisture wicking properties can be knitted, woven or non-woven textiles. In some embodiments, these textiles are located in a position towards the outer surface of a textile and/or garment to allow the absorbed moisture to easily evaporate.

The RPT can also be constructed from filament yarn, or spun yarn, or blended yarn comprising recombinant protein fibers with properties described in the present disclosure. Structures of yarns comprising RPFs are described in International Publication No. WO2016/201369, which is incorporated by reference herein in its entirety.

In some embodiments, the RPTs described herein comprise RPYs that are filament yarns, or spun yarns, or blended yarns. In some embodiments, the RPTs described herein comprise RPYs with linear density less than 5 dtex, or less than 3 dtex, or less than 2 dtex, or less than 1.5 dtex, or greater than 1.5 dtex, or greater than 1.7 dtex, or greater than 2 dtex, or from 1 to 5 dtex, or from 1 to 3 dtex, or from 1.5 to 2 dtex, or from 1.5 to 2.5 dtex.

In some embodiments, the RPT before or after pre-treatment comprises RPYs that have an average or median fiber twist of 5 to 20 twists per inch (TPI), or from 5 to 15 TPI, or from 5 to 10 TPI, or from 8 to 10 TPI.

In some embodiments, the RPT comprises RPYs that are plied. In some embodiments, the RPT comprises 2 or more RPYs that are plied using an S twist to create a first set of yarns, and then plying the S twisted yarns together using a Z twist to form the plied yarn.

In some embodiments, the RPTs of the present disclosure comprise RPYs comprising an outer sheath comprising the recombinant protein fiber, wherein the outer sheath comprises a greater twist as compared to a twist in a center core of the filament yarn, and optionally wherein the mean denier of the recombinant protein fiber is less than 2, or less than 5, or less than 10.

In some embodiments, the RPTs of the present disclosure comprise yarns comprising a blended yarn comprising RPFs and fibers selected from the group consisting of cotton, wool, merino, mohair, polyamide, linen, acrylic, polyester, spandex, and combinations thereof.

In some embodiments, the RPTs of the present disclosure contain RPYs with desirable properties (e.g., strength, modulus, extensibility, moisture management). In some embodiments, the RPTs of the present disclosure contain RPYs with desirable physical properties, mechanical properties, moisture properties, antimicrobial properties, cross-section shape, other properties, and combinations of properties. RPY physical properties, mechanical properties, moisture properties, antimicrobial properties, cross-section shape, other properties, and combinations of properties are described in International Publication No. WO 2016/201369, incorporated by reference herein in its entirety.

In some embodiments, the RPTs of the present disclosure comprise RPFs with desirable physical properties, mechanical properties, moisture properties, antimicrobial properties, cross-section shape, other properties, and combinations of properties. RPF physical properties, mechanical properties, moisture properties, antimicrobial properties, cross-section shape, other properties, and combinations of properties are described in International PCT Publications WO 2014/066374, WO 2015/042164, WO 2016/149414, and WO 2016/201369, each of which are incorporated by reference herein in their entirety.

In some embodiments, RPTs on the present disclosure comprise RPFs, wherein the RPFs comprise proteins that comprise repeat units, wherein each repeat unit has at least 99% sequence identity to a sequence that comprises from 2 to 20 quasi-repeat units, each quasi-repeat unit having a composition comprising [GGY-{GPG-X₁]n₁-GPS-(A)n₂], wherein for each quasi-repeat unit: X₁ is independently selected from the group consisting of SGGQQ, GAGQQ, GQGPY, AGQQ, and SQ; and n₁ is from 4 to 8, and n₂ is from 6 to 10.

In some embodiments, RPTs on the present disclosure comprise RPFs, wherein the RPFs comprise proteins comprising 2 or more concatenated repeats of SEQ ID NO: 1, or circularly permuted variants thereof.

TABLE 1
Example sequence for proteins in RPTs

| Seq. | |
|---|---|
| ID | |
| No. | AA Sequence |
| 1 | GGYGPGAGQQGPGSGGQQGPGGQGPYGSGQQGPGGAGQQGPGGQ |
| | GPYGPGAAAAAAAAAGGYGPGAGQQGPGGAGQQGPGSQGPGGQG |
| | PYGPGAGQQGPGSQGPGSGGQQGPGGQGPYGPSAAAAAAAAAGG |
| | YGPGAGQRSQGPGGQGPYGPGAGQQGPGSQGPGSGGQQGPGGQG |
| | PYGPSAAAAAAAAGGYGPGAGQQGPGSQGPGSGGQQGPGGQGPY |
| | GPGAAAAAAAVGGYGPGAGQQGPGSQGPGSGGQQGPGGQGPYGP |
| | SAAAAAAAAGGYGPGAGQQGPGSQGPGSGGQQGPGGQGPYGPSA |
| | AAAAAAA |

METHODS OF PRODUCING AND PRE-TREATING RPTS

In some embodiments, yarns comprising recombinant protein fibers are manufactured into textiles (i.e., RPTs), for example by weaving or knitting. In some embodiments, RPFs are manufactured into RPTs by knitting using a circular knitting apparatus, a warp knitting apparatus, a flat knitting apparatus, a one piece knitting apparatus, or a 3-D knitting apparatus. In some embodiments, RPFs are manufactured into RPTs by weaving using a plain weave loom, a dobby loom or a jacquard loom. In some embodiments, RPFs are manufactured into RPTs using a 3d printing method. In some embodiments, RPFs are manufactured into non-woven RPTs using techniques such as wet laying, spin bonding, stitch bonding, spunlacing (i.e., hydroentanglement), or needlepunching. In embodiments, the RPT construction, the RPY structure and the RPF properties are chosen to impart various characteristics to the resulting yarns and textiles.

Methods of producing RPYs comprising RPFs are described in International Publication No. WO 2016/201369 A1 (“Recombinant protein fiber yarns with improved properties”), which is incorporated by reference herein in its entirety.

Methods of producing RPFs are described in International PCT Publications WO 2014/066374, WO 2015/042164, WO 2016/149414, and WO 2016/201369, each of which are incorporated by reference herein.

In some embodiments, methods of pre-treating RPTs include exposing the RPT to moisture. In some embodiments, methods of pre-treating RPTs by exposing the RPT to moisture has the effect of shrinking the RPTs (i.e., reducing one or more physical dimension of the RPT). In some embodiments, methods of pre-treating RPTs include submerging the RPT in water. In some embodiments, methods of pre-treating RPTs by submerging the RPT in water has the effect of shrinking the RPTs (i.e., reducing one or more physical dimension of the RPT).

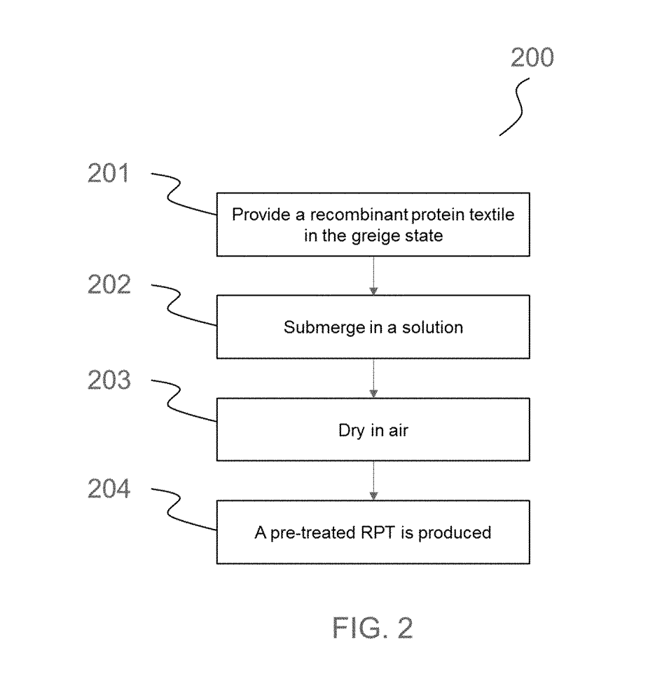

FIG. 2 illustrates a method to pre-treat RPTs, in some embodiments. In some embodiments, a method to pre-treat RPTs comprises the following steps: provide an RPT (step 201 in FIG. 2), submerge the RPT in a solution (step 202 in FIG. 2), and dry in air (step 203 in FIG. 2). The result of performing steps 201, 202 and 203 is that a pre-treated RPT is produced (204 in FIG. 2). In some embodiments, the solution (in step 202 in FIG. 2) is water, or is greater than 99% water, or is greater than 90% water. In some embodiments, the solution (in step 202 in FIG. 2) is water, alcohol (e.g., methanol, ethanol, etc.), or is a mixture of water and alcohol. In some embodiments, the solution (in step 202 in FIG. 2) comprises one or more additives in addition to water, alcohol (e.g., methanol, ethanol, etc.), or a mixture of water and alcohol. In some embodiments, the additive in the solution (in step 202 in FIG. 2) comprises lubricating additives, cohesive additives, surfactants, or other additives to assist the RPT shrinking in the pre-treatment step. Some examples of additives include coconut oil and esters of coconut fatty acids, esters of steric acid, esters of myristic acid, petroleum based lubricants such as mineral oil, alcohol ether ethoxylates (and derivatives thereof), derivatives of fatty acid esters of ethylene oxide, silicones. In some embodiments, the RPT provided (in step 201 in FIG. 2) is knitted, or woven, or non-woven. In some embodiments, the step wherein the RPT is submerged in the solution (step 202 in FIG. 2) is performed at a temperature from 15° C. to 50° C., or from 15° C. to 45° C., or from 15° C. to 40° C., or from 15° C. to 35° C., or from 15° C. to 30° C., or from 15° C. to 25° C., or at approximately 20° C. (i.e., room temperature). In some embodiments, the step wherein the RPT is submerged in solvent (step 202 in FIG. 2) is performed without applying tension. In some embodiments, the step wherein the RPT is dried in air (step 203 in FIG. 2) is performed at a temperature from 15° C. to 50° C., or from 15° C. to 45° C., or from 15° C. to 40° C., or from 15° C. to 35° C., or from 15° C. to 30° C., or from 15° C. to 25° C., or at approximately 20° C. (i.e., room temperature). In some embodiments, the step wherein the RPT is dried in air (step 203 in FIG. 2) is performed without applying tension.

In alternate embodiments, step 202 in the method shown in FIG. 2 could be performed by exposing the RPT to moisture by dampening, moisturizing, steaming, or exposing said garment to humid conditions. In some embodiments, step 202 in the method shown in FIG. 2 could be performed by exposing the RPTs to a solution (e.g., water, or a solution that is greater than 99% water, or a solution that is greater than 90% water) uniformly.

In alternate embodiments, step 202 and/or 203 in the method shown in FIG. 2 could be performed while a tension is applied to the RPT. In some embodiments, the tension applied is from 1 to 100 MPa, or from 20 to 80 MPa, or from 30 to 70 MPa, or from 40 to 60 MPa, or greater than 60 MPa, or greater than 100 MPa.

The amount of shrinkage in an RPT will depend on the properties of the RPFs, the construction of the yarns (e.g., twist per inch, and ply), and the construction of the RPT. For example, a knitted RPT with a particular knitted pattern may not have the same amount of shrinkage during pre-treatment as a knitted RPT with a different knitted pattern. Some examples of factors that can affect the amount of shrinkage in an RPT during pre-treatment include the RPF composition, the RPF linear density, the RPY linear density, the number of RPFs in the RPY, the RPY twist, the RPY ply, and the RPT construction (e.g., the RPT knitted or woven pattern).

In some embodiments, the RPT shrinks during pre-treatment by from 5% to 50%, or from 10% to 50%, or from 15% to 50%, or from 20% to 50%, or from 25% to 50%, or from 5% to 40%, or from 10% to 40%, or from 15% to 40%, or from 20% to 40%, or from 25% to 40%.

In some embodiments the RPT shrinks during pre-treatment by different amounts in the width and length, wherein the amount of shrinkage in the width, or the length, is from 5% to 50%, or from 10% to 50%, or from 15% to 50%, or from 20% to 50%, or from 25% to 50%, or from 5% to 40%, or from 10% to 40%, or from 15% to 40%, or from 20% to 40%, or from 25% to 40%. In some embodiments the RPT shrinks during pre-treatment by different amounts in the width and length, wherein the ratio of the amount of shrinkage in the width to the amount of shrinkage in the length (i.e., a ratio between two orthogonal dimensions) is from 0 to 2, or from 0.5 to 2, or from 1 to 2, or from 1.5 to 2, or from 0 to 1.5, or from 0.5 to 1.5, or from 1 to 1.5, or from 0 to 1, or from 0.5 to 1.

In some embodiments, the RPT is knitted and there is a different amount of shrinkage in the wale direction and the course direction, wherein the amount of shrinkage in the wale direction, or the course direction, is from 5% to 50%, or from 10% to 50%, or from 15% to 50%, or from 20% to 50%, or from 25% to 50%, or from 5% to 40%, or from 10% to 40%, or from 15% to 40%, or from 20% to 40%, or from 25% to 40%.

In some embodiments, the RPT is woven and there is a different amount of shrinkage in the warp direction and the weft direction, wherein the amount of shrinkage in the warp direction, or the weft direction, is from 5% to 50%, or from 10% to 50%, or from 15% to 50%, or from 20% to 50%, or from 25% to 50%, or from 5% to 40%, or from 10% to 40%, or from 15% to 40%, or from 20% to 40%, or from 25% to 40%.

In some embodiments the RPT shrinks during pre-treatment by different amounts in the course and wale of a knitted RPT, or in the warp and weft of a woven RPT, wherein the ratio of the amount of shrinkage in the warp or course to the amount of shrinkage in the weft or wale (i.e., a ratio between two orthogonal dimensions) is from 0 to 2, or from 0.5 to 2, or from 1 to 2, or from 1.5 to 2, or from 0 to 1.5, or from 0.5 to 1.5, or from 1 to 1.5, or from 0 to 1, or from 0.5 to 1.

In some embodiments, the area of the RPT decreases during pre-treatment by from 10% to 75%, or from 20% to 75%, or from 30% to 75%, or from 40% to 75%, or from 50% to 75%, or from 10% to 60%, or from 20% to 60%, or from 30% to 60%, or from 40% to 60%, or from 50% to 60%.

In some embodiments, an RPT is constructed such that different sections of the RPT will shrink differently, and after pre-treatment the RPT will have an induced shape or texture. One example is an RPT that has alternating stripes of jersey knit and a decorative knit, where the jersey knitted sections shrink more than the decorative knitted sections. After pre-treatment, the RPT would have a ridged texture due to the differential shrinking of the different sections. Another example is a flat-knitted RPT with a central region of decorative knit surrounded by a border that has a jersey knitted pattern, where the jersey knitted sections shrink more than the decorative knitted sections. After pre-treatment, the border of the RPT will shrink more than the center, which can cause the RPT to form a 3D shape where the center bulges up above the plane formed by the border.

In some embodiments, additional treatments can be applied to the RPTs before or after the pre-treatment processes described herein. Some examples of other treatments that can be applied to the RPTs along with the pre-treatment processes describe herein, are the application of a finish (e.g., a moisture sealant, a fire-retardant, an anti-bacterial coating), a flattening or ironing step (e.g., using rollers), or the application of a volatile substance (e.g., a fragrance). In some embodiments, treatments that will be affected by moisture or that will impact the RPF interaction with moisture, such as the application of volatile substances or moisture sealants, are applied after the pre-treatment processes described herein.

In some embodiments, the RPTs are produced and stored in a humidity controlled environment during knitting and after knitting before pre-treatment. In some embodiments, the humidity is controlled to a relative humidity of 25%, or 30%, or 35%, or 40%, or 45%, or 50%, or 55%, or 60%, or 65%, or 70%, or 75% during knitting and after knitting before pre-treatment. Exposure to moisture while the RPFs are unconstrained, or in a low tension state, can result in shrinking during knitting, and affect the degree of shrinking during pre-treatment.

### METHODS OF PRODUCING AND PRE-TREATING GARMENTS INCLUDING RPTS

In some embodiments, a garment is produced from the RPTs described herein. In some embodiments, a garment is produced from RPTs, and pre-treated using the compositions and methods described herein. In some embodiments, the supercontraction of the RPFs making up the RPTs in the garment causes the garment to shrink when pre-treated (e.g., exposed to moisture) with the same affecting variables and final product effects as described for RPTs herein. Additionally, garments may be made up of different RPTs in different sections of the garment causing uneven shrinking of the garment during pre-treatment.

In some embodiments, garments are produced from knitted RPTs. In some embodiments, a garment containing RPTs is produced by knitting a full garment (e.g., a hat), or by knitting followed by linking or sewing (e.g., a necktie), or by knitting then cutting a pattern and then sewing the cut pieces together (e.g., a tee shirt).

In some embodiments, a garment is produced from an RPT using the compositions and constructions described herein, and pre-treated using the methods described herein. In some embodiments, the garment includes knitted PRTs. In some embodiments, the knit tension in the knitting machine is calculated using the known shrinkage of the yarns in the course and wale directions, and the RPTs are under-constructed by adding the percent of shrinkage to the dimension of the knit program in each direction in order to produce a textile that would finish to the correct size once it underwent the pre-treating process.

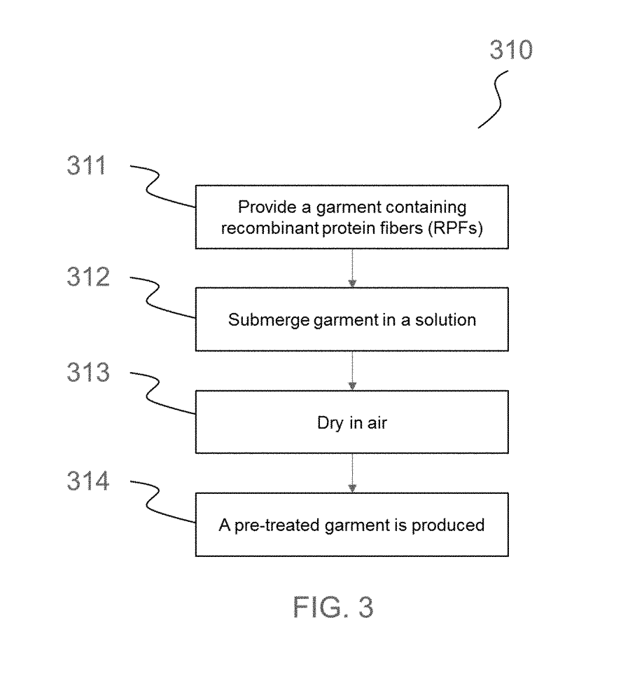

FIG. 3 illustrates a method to pre-treat garments containing RPTs, in some embodiments. In some embodiments, a method to pre-treat a garment comprising RPTs is to provide a garment comprising RPTs (step 311 in FIG. 3), submerge the garment comprising RPTs in a solution (step 312 in FIG. 3), and dry the garment in air (step 313 in FIG. M2). Some examples of the garment comprising RPTs in the method above is a shirt, or pants, or a sock, or a hat, or a necktie, or underwear. In some embodiments, the solution (in step 312 in FIG. 3) is water, or is greater than 99% water, or is greater than 90% water, or is greater than 80% water, or is greater than 70% water, or is greater than 60% water. In some embodiments, the solution (in step 312 in FIG. 3) is water, alcohol (e.g., methanol, ethanol, etc.), or is a mixture of water and alcohol. In some embodiments, the solution (in step 312 in FIG. 3) comprises one or more additives in addition to water, alcohol (e.g., methanol, ethanol, etc.), or a mixture of water and alcohol. In some embodiments, the additive in the solution (in step 312 in FIG. 3) comprises lubricating additives, cohesive additives, surfactants, or other additives to assist the RPT shrinking in the pre-treatment step. Some examples of additives include coconut oil and esters of coconut fatty acids, esters of steric acid, esters of myristic acid, petroleum based lubricants such as mineral oil, alcohol ether ethoxylates (and derivatives thereof), derivatives of fatty acid esters of ethylene oxide, silicones. In some embodiments, the method above includes a garment containing RPTs that are knitted, or woven, or non-woven. In some embodiments, the step wherein the garment containing RPTs is submerged in the solution is performed at a temperature from 15° C. to 50° C., or from 15° C. to 45° C., or from 15° C. to 40° C., or from 15° C. to 35° C., or from 15° C. to 30° C., or from 15° C. to 25° C., or at approximately 20° C. (i.e., room temperature). In some embodiments, the step wherein the garment containing RPTs is submerged in solvent is performed without applying tension. In some embodiments, the step wherein the garment containing RPTs is dried in air is performed at a temperature from 15° C. to 50° C., or from 15° C. to 45° C., or from 15° C. to 40° C., or from 15° C. to 35° C., or from 15° C. to 30° C., or from 15° C. to 25° C., or at approximately 20° C. (i.e., room temperature). In some embodiments, the step wherein the garment containing RPTs is dried in air is performed without applying tension.

In alternate embodiments, step 312 in the method shown in FIG. 3 could be performed by exposing the garment containing RPTs to moisture by dampening, moisturizing, steaming, or exposing said garment to humid conditions. In some embodiments, step 312 in the method shown in FIG. 3 could be performed by exposing the garment containing RPTs to a solution (e.g., water, or a solution that is greater than 99% water, or a solution that is greater than 90% water) uniformly.

In alternate embodiments, step 312 and/or 313 in the method shown in FIG. 3 could be performed while a tension is applied to the garment containing RPTs. In some embodiments, the tension applied is from 1 to 100 MPa, or from 20 to 80 MPa, or from 30 to 70 MPa, or from 40 to 60 MPa, or greater than 60 MPa, or greater than 100 MPa.

In some embodiments, the garment includes knitted RPTs, and the knitted RPTs have different patterns in different sections of the garment. In some embodiments, the tension and degree of under-construction is varied for each section of the garment, to account for the different sections shrinking by different amounts during pre-treatment. Therefore, before pre-treatment, the different sections of the garment can have different widths (i.e., distance across the garment when laid flat) at the interface between sections. After pre-treatment, the sections of the garment can shrink and conform to the same widths so that there are no width changes from section to section in the garment.

In some embodiments, the garment containing RPTs is placed on a mold during pre-treatment. One example of a mold is a mannequin in the shape of a person. In some embodiments, the mold could assist the garment to shrink to a particular shape. In some embodiments, after the pre-treatment using a mold, a method would be applied to stop the RPTs in the garment from shrinking further (e.g., a moisture sealant could be applied).

In some embodiments, a garment containing RPTs is constructed such that different sections of the garment will shrink differently, and after pre-treatment the garment will have an induced shape or texture. One example is a garment containing RPTs that have alternating stripes of jersey knit and a decorative knit, where the jersey knitted sections shrink more than the decorative knitted sections. After pre-treatment, the garment would have a ridged texture due to the differential shrinking of the different sections. Another example is a garment containing a flat-knitted RPT with a central region of decorative knit surrounded by a border that has a jersey knitted pattern, where the jersey knitted sections shrink more than the decorative knitted sections. After pre-treatment, the border of the RPT will shrink more than the center, which can cause the RPT to form a 3D shape where the center bulges up above the plane formed by the border. Such methods could be used, for example, to produce garments with loft added to elbow or knee regions.

### EXAMPLES

### Example 1

### Necktie with Pre-Treated RPTs

A necktie (i.e., a tie) was produced from RPTs, and pre-treated.

The necktie in this example was produced with RPFs comprising copolymers that were secreted from Pichia pastoris commonly used for the expression of recombinant DNA using published techniques, i.e., those described in International Publication No. WO 2015/042164, at paragraphs 114-134, incorporated herein by reference. The copolymer polypeptide utilized for fiber spinning in this Example was SEQ ID NO. 1, concatenated 3 times, with a 3× FLAG sequence: GDYKDDDDKDYKDDDDKDYKDDDDK (SEQ ID NO: 200) bound to the C-terminal end of the polypeptide. The secreted proteins were purified and dried using standard techniques. The dried polypeptide powder was dissolved in a formic acid-based spinning solvent to generate a homogenous spin dope.

The RPFs in this example were spun by extruding the spin dope through a 50 orifice spinneret with 75 μm diameter orifices with 2:1 ratio of length to diameter into a room temperature alcohol-based coagulation bath with a residence time of approximately 15 seconds. Fibers were pulled out of the coagulation bath under tension, and then drawn to approximately 6 times their length, and dried. The volumetric flow rate out of spinneret was approximately 1.25 mL/min.

The necktie in this example was produced with RPYs made from the RPFs described above using a twisting and plying technique. Two tows were lubricated off the line and twisted using an S twist, and then plied using a Z twist. The twist applied was approximately 8-10 twists per inch. The RPYs were produced on a semi-worsted spinning frame. No additional finish was applied to the RPYs.

The RPFs used to produce the necktie in this Example had an average linear density of approximately 5.7 dtex. The RPYs used to produce the necktie in this Example had an average linear density of approximately 550 to 600 dtex, an average tenacity of approximately 9 cN/tex, and an average initial modulus of approximately 300 to 400 cN/tex.

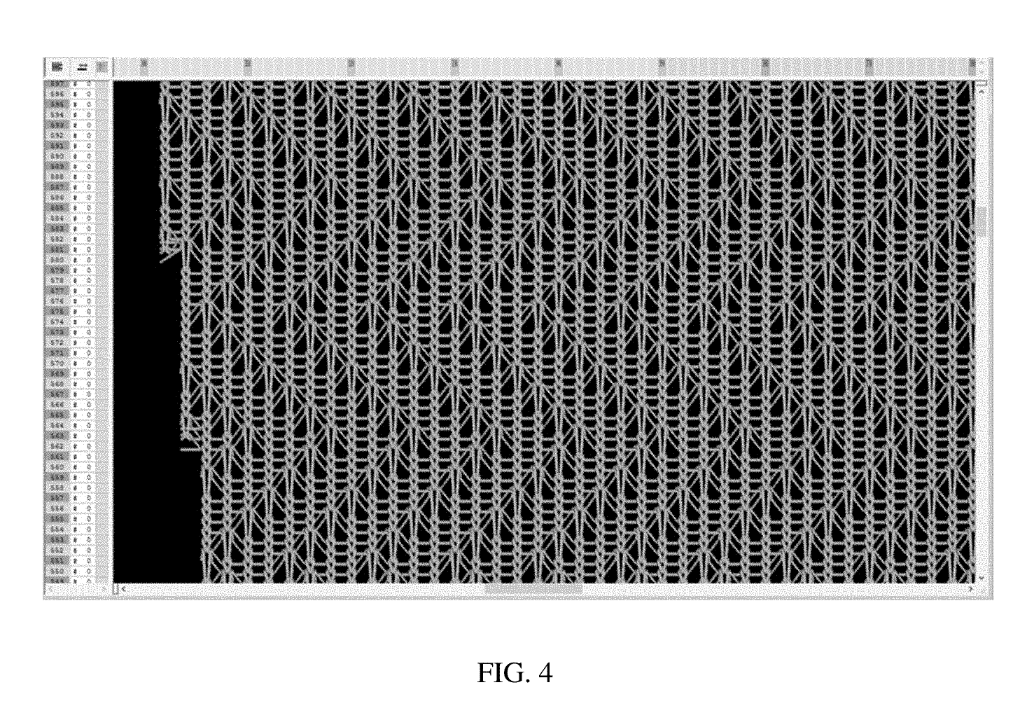

The RPTs for the necktie in this example were produced by knitting using an industrial knitting machine. The necktie was knitted inside-out in one continuous tube, and is then pulled through after knitting and the ends were linked to close them. The necktie comprised 4 sections, a back-neck section mainly behind the neck of the wearer, a knot section where the knot is tied, a main section making up the bulk of the visible section of the tie when it is worn, and a stripe section at the lowest portion of the tie when it is worn. The necktie comprises different knit patterns in the different sections of the garment. The main section of the necktie comprised an RPT with a decorative pattern. The decorative pattern of the necktie has a base of a reverse jersey with zig-zags creating a reverse herringbone pattern, which were created with a combination of a tuck stitch and a jersey stitch. The repeat length of the decorative pattern is 24 courses: the herringbone zig-zags proceeded in one direction for 12 courses, and then back the opposite direction for 12 courses, and then the pattern repeats. FIG. 4 illustrates a representation of the decorative pattern used in the necktie (note that the stitch density is not to scale). The back neck section of the necktie, the section of the necktie where the knot is tied, and the stripe section all contained RPTs with a simple reverse jersey knitted pattern. For all of the sections of the necktie, the knit tension in the knitting machine was calculated using the known shrinkage of the yarns in the course and wale directions. The RPTs were under-constructed by adding the percent of shrinkage to the dimension of the knit program in each direction in order to produce a textile that would finish to the correct size once it underwent the pre-treating process. In this example, the tension and degree of under-construction varied for each section of the necktie, because the reverse jersey knitted sections shrank more during pre-treatment than the sections with the decorative pattern. Therefore, before pre-treatment, the different sections of the necktie were different widths (i.e., distance across the tie when laid flat) at the interface between sections. After pre-treatment, the sections of the necktie shrank differentially, and the abrupt changes in width from section to section in the necktie were reduced.

The tie comprising the under-constructed RPTs was pre-treated by submerging in water for approximately 5 minutes at approximately 22° C. The tie comprising RPTs was then laid flat to dry in air at approximately 22° C. and approximately 65% relative humidity.

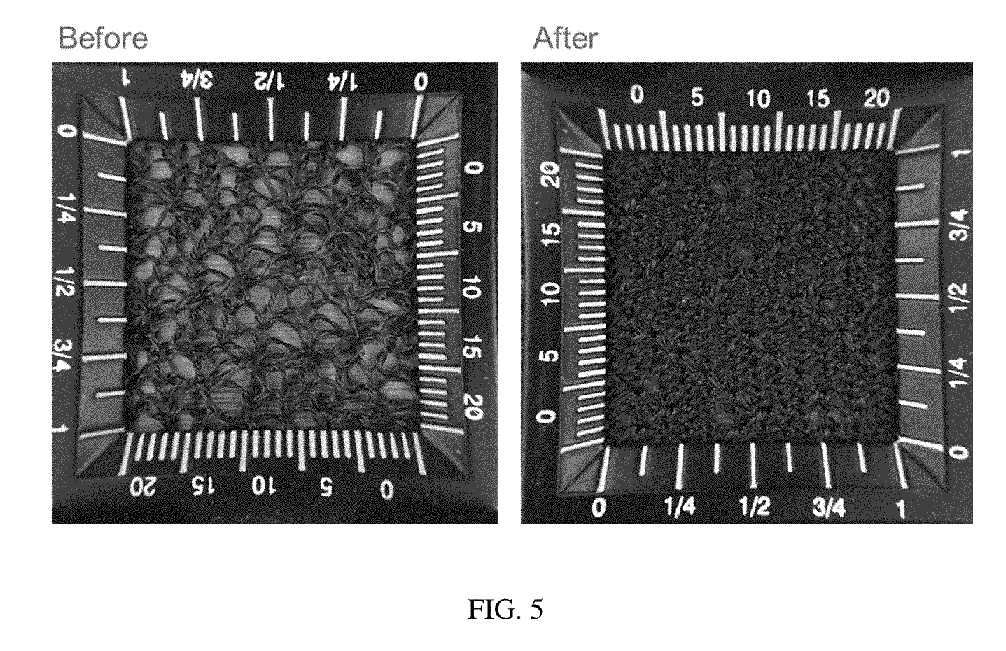

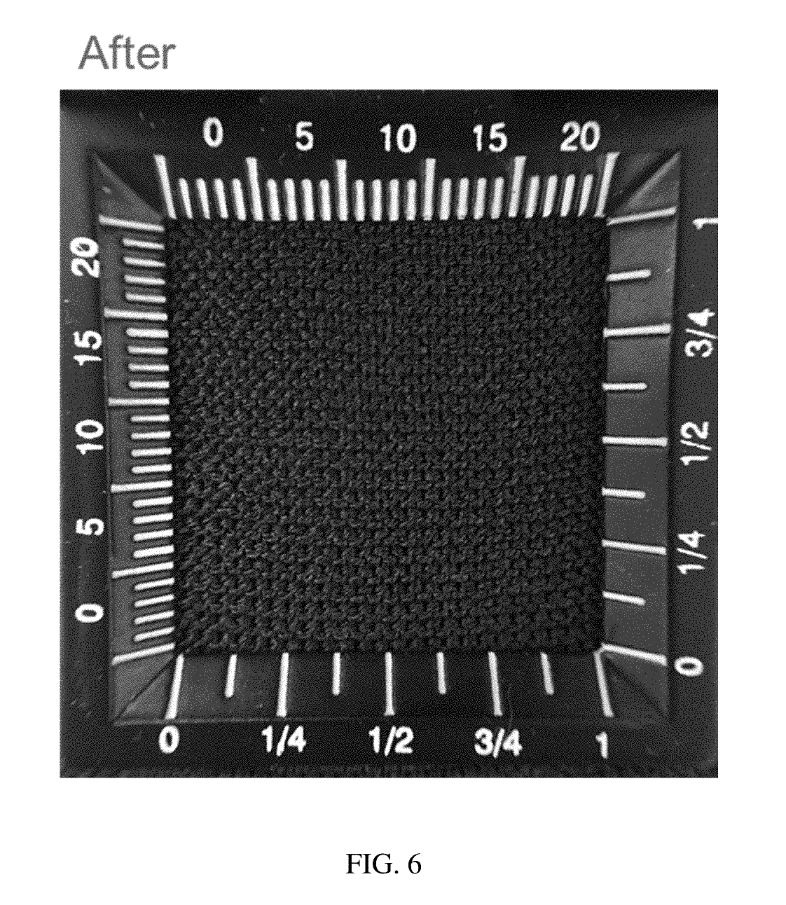

Upon pre-treatment, the RPFs comprising the necktie in this example shrank by approximately 30%, and the RPTs comprising the necktie in this example shrank by approximately 40% in the wale direction and 20% in the course direction. FIG. 5 shows an RPT with the same knitted pattern as the decorative portion of the before and after pre-treatment. The figure shows that the stitches per inch (SPI) in the course direction changed from about 10 to about 15 SPI during pre-treatment. After pre-treatment, the RPT had a stitch density that was within the acceptable range, which resulted in a desirable drapability. FIG. 6 shows an RPT with the same knitted pattern as the reverse jersey knitted portion of the tie after pre-treatment. After pre-treatment, the RPT had a stitch density that was within the acceptable range, which resulted in a desirable drapability.

After pre-treatment, the RPTs comprising the necktie in this example had a character of textile hand that was achieved, had a high degree of drape, and had a tough texture. After pre-treatment, the RPTs comprising the necktie in this example had some luster which could be described as bright.

### Example 2

### Pre-Treating RPFs and RPTs

This Example describes results from experiments where RPFs and RPTs were provided and pre-treated. The RPFs in this Example were produced using the same methods described in Example 1. The pre-treatment for all of the samples in this Example was performed by submerging the sample in water for approximately 5 minutes at approximately 22° C. The sample was then laid flat to dry in air at approximately 22° C. and approximately 65% relative humidity.

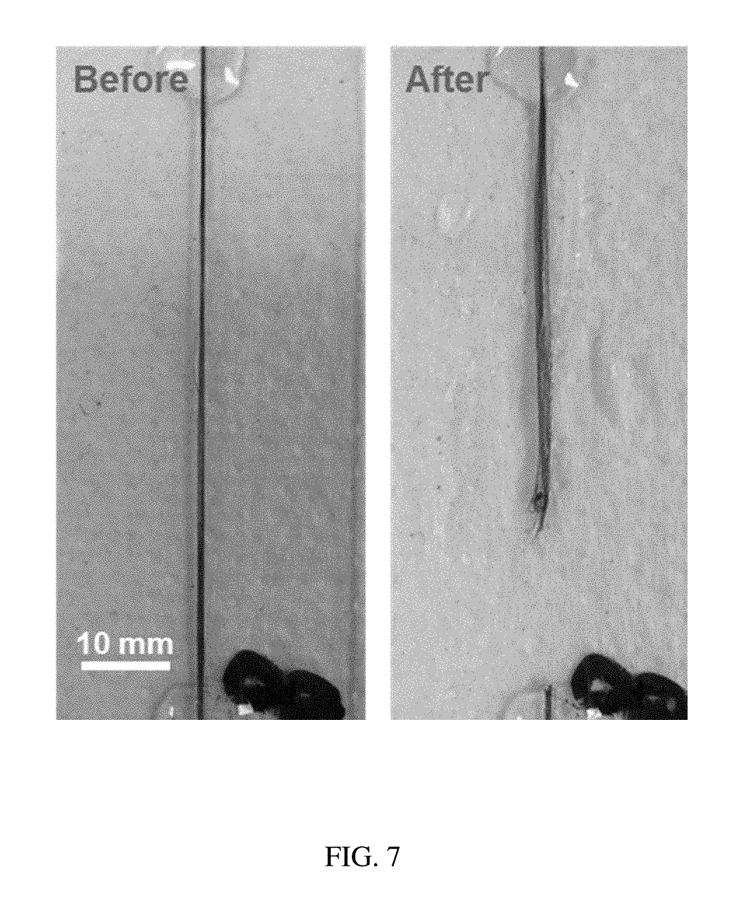

FIG. 7 shows 2 photographs of a tow of 50 RPFs before and after pre-treatment. The tow of RPFs in this Example supercontracted by approximately 25%, or from 20 to 30%.

A series of RPTs with different stitch density were constructed, and then pre-treated by submerging in water. The RPFs used in the RPTs in this example followed the same procedure described for the RPTs used in Example 1. The RPYs used in the RPTs in this Example were made from tows of 50 fibers, and were either twisted filament yarns (with 1 end), or were plied filament yarns with 2 ends. The RPTs in this Example were produced using an industrial knitting machine, and have a simple jersey knitted pattern.

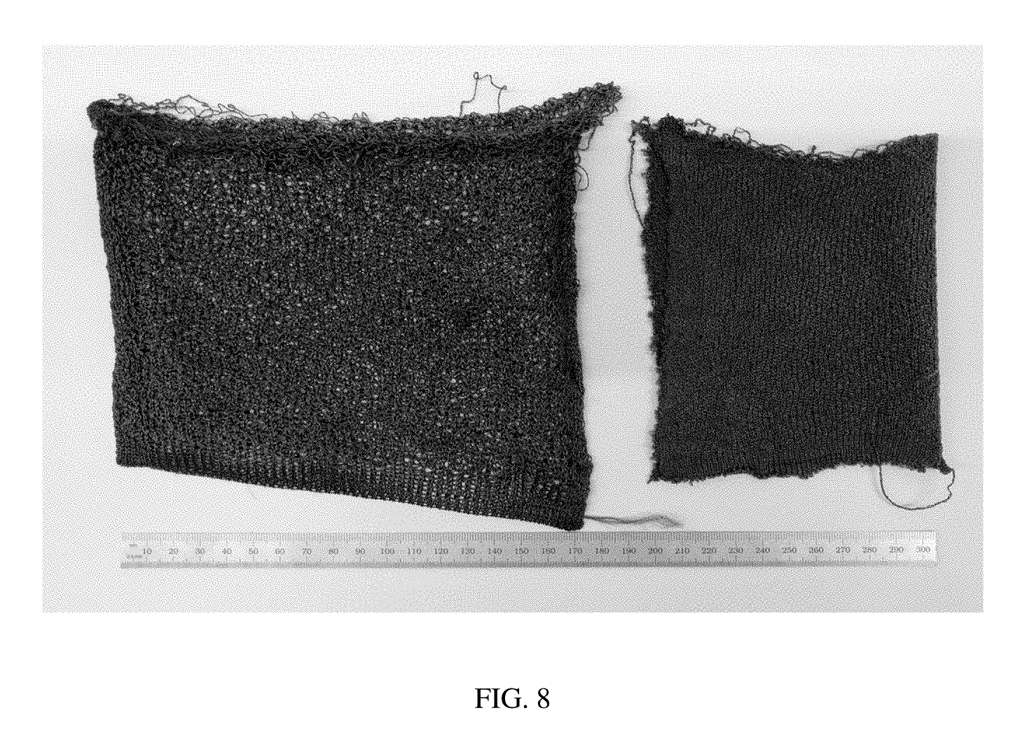

FIG. 8 shows a photograph of an RPT before pre-treatment, and an RPT after pre-treatment. The RPTs shown in this figure have a simple jersey knit pattern. The figure shows that the jersey knitted RPT shrinks more in the wale direction than the course direction.

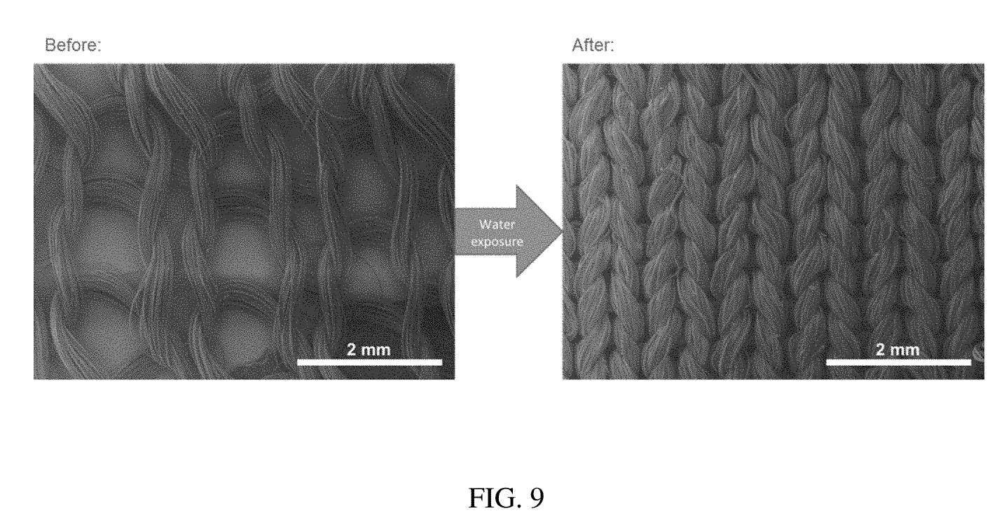

FIG. 9 shows a scanning electron microscope (SEM) image of a knitted RPT before pre-treatment, and an RPT after pre-treatment. The RPTs shown in this figure have a simple jersey knit pattern. The figure illustrates that the RPT is under-constructed by a considerable amount before pre-treatment, and after pre-treatment the RPT has a desirable knitted construction. The figure illustrates that the RPT before pre-treatment has fewer stitches per unit length than the RPT after pre-treatment.

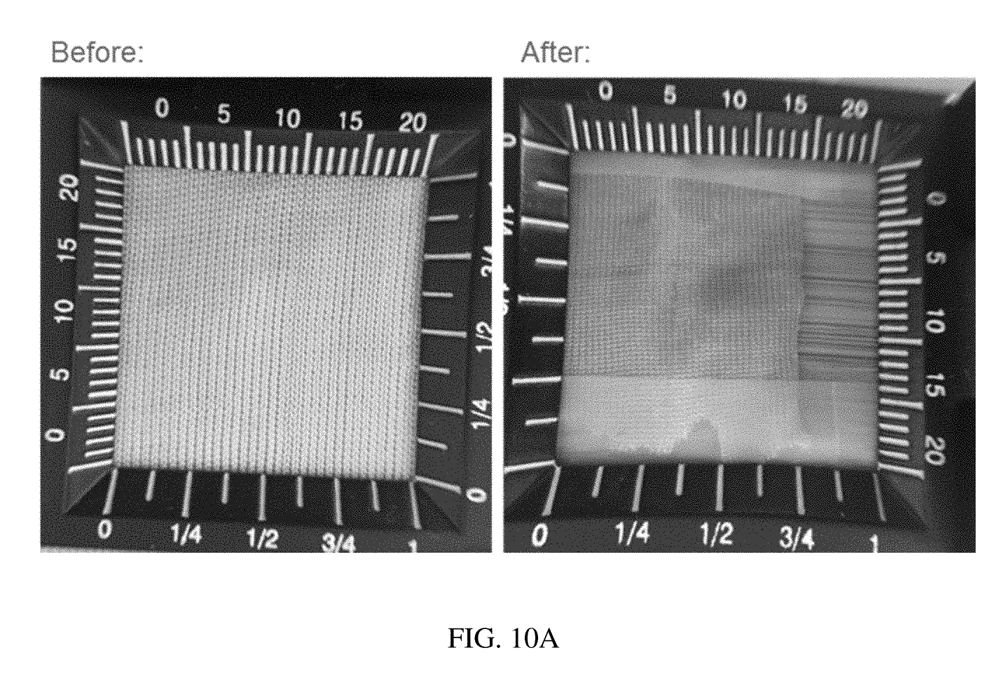

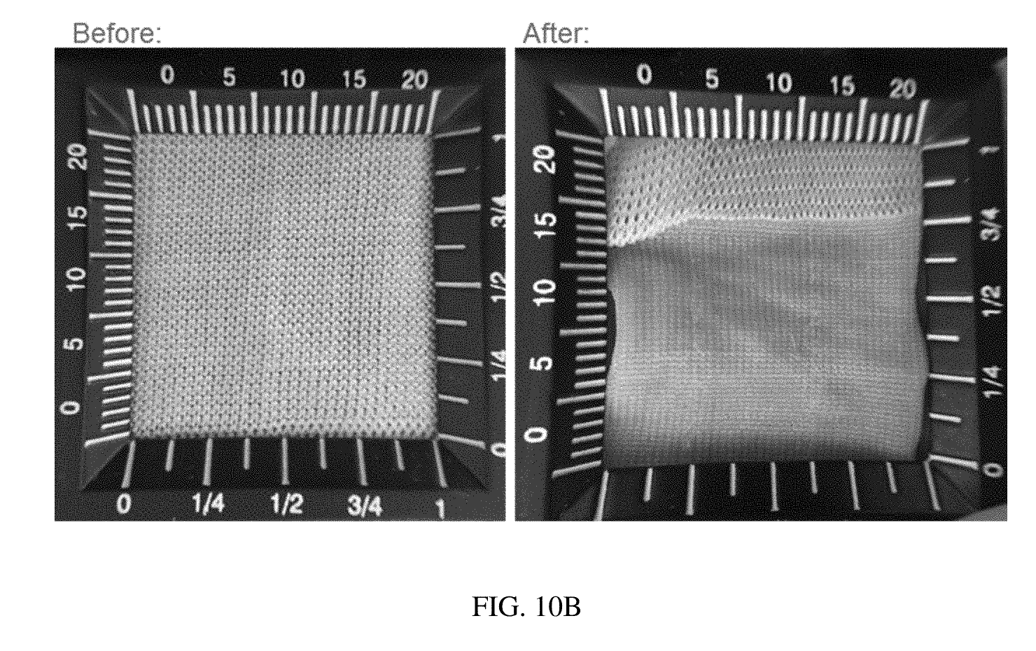

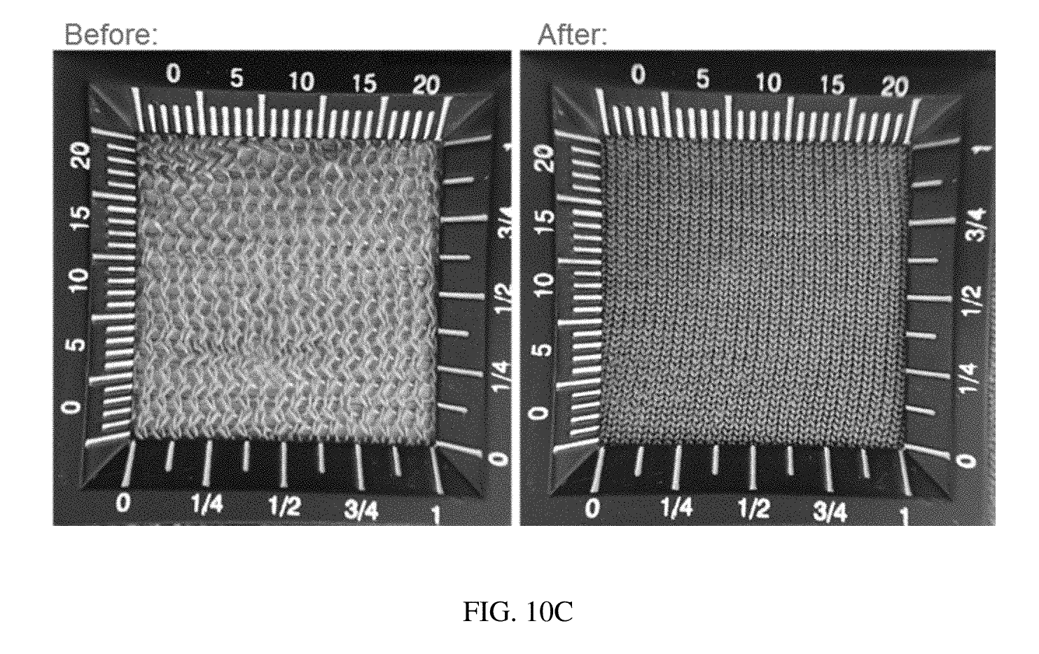

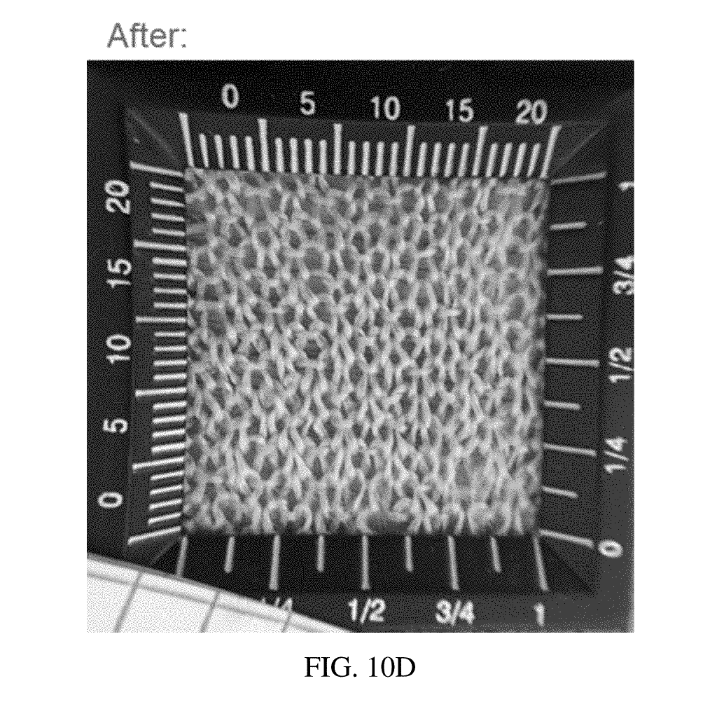

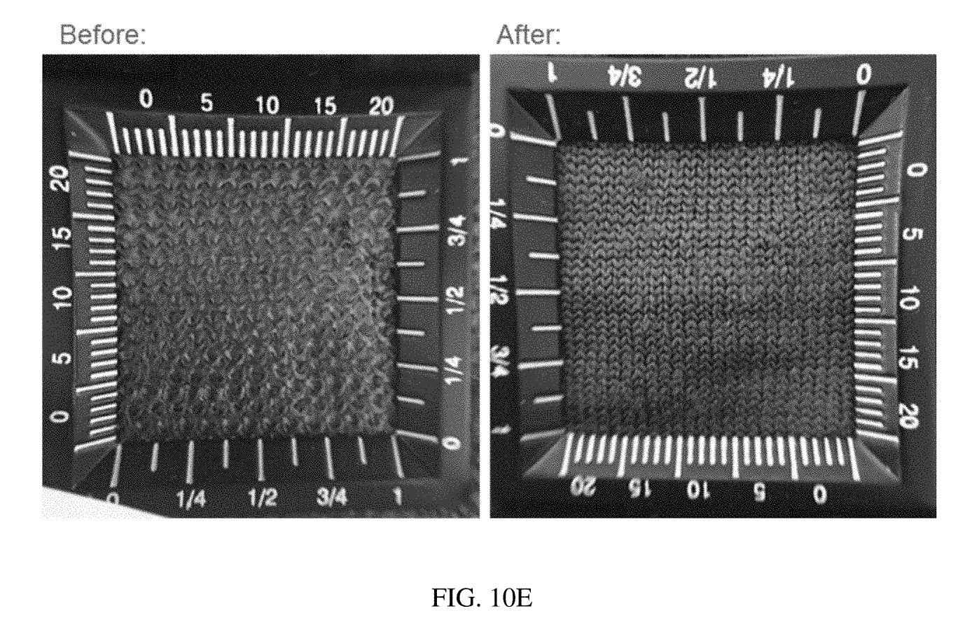

A number of experiments were performed to evaluate how knitted RPTs with different greige state constructions behaved before, during and after pre-treatment. This information was gathered empirically due to the large degree of shrinking during pre-treatment, and the complex nature of the change in construction for different knitted RPTs as the constituent RPFs supercontract during pre-treatment. Table 2 summarizes some of the results of these tests on jersey knitted RPTs. Pre-treatment was performed by submerging each RPT in water for approximately 5 minutes at approximately 22° C. The RPTs were then laid flat to dry in air at approximately 22° C. and approximately 65% relative humidity. The table describes the number of ends (i.e., the number of tows in the plied yarn), the stitch linear density in the course and wale direction before and after pre-treatment, the percentage of shrinkage in the course and wale directions resulting from pre-treatment, and an assessment of the drapability of the RPT after pre-treatment (i.e., in the contracted state). FIGS. 10A to 10E show photographs of each RPT in Table 2 before and after pre-treatment including horizontal and vertical scale bars in centimeters and inches to aid in determination of the stitch density.

TABLE 2
Experimental results for jersey knitted RPTs before and after pre-treatment.

| | | Course - | Course - | Shrinkage | Wale - | Wale | Shrinkage | |
|---|---|---|---|---|---|---|---|---|
| | | before | after | in the course | before | after | in the wale | Contracted |
| Number | | [stiches/ | [stiches/ | direction | [stiches/ | [stiches/ | direction | textile |
| of ends | FIG. | inch] | inch] | [%] | inch] | inch] | [%] | drapability |
| one | 10A | 34 | 52 | 35 | 45 | 74 | 39 | Brittle |
| one | 10B | 31 | 43 | 28 | 32 | 64 | 50 | Rope-like |
| one | 10C | 17 | 28 | 39 | 21 | 47 | 55 | Desirable |
| one | 10D | 7 | 14 | 50 | 15 | 13 | −15* | Unstructured |
| two | 10E | 13 | 23 | 43 | 15 | 33 | 55 | Rope-like |

FIG. 10A shows an RPT before and after pre-treatment that was not under-constructed enough before pre-treatment. The stitch density in the course direction, for example, changed from 34 SPI to 52 SPI during pre-treatment. The resulting RPT had a stitch density that was too high, which resulted in an undesirable drapability that was classified as brittle. In this context brittle indicates that bending the textile resulted in fibers breaking and sharp creases being formed.

FIG. 10B shows an RPT before and after pre-treatment that was also not under-constructed enough before pre-treatment. The stitch density in the course direction, for example, changed from 31 SPI to 43 SPI during pre-treatment. The resulting RPT had a stitch density that was too high, which resulted in an undesirable drapability that was classified as rope-like. In this context rope-like indicates that the textile resisted a bending force and did not want to follow contours of the body.

FIG. 10C shows an RPT before and after pre-treatment that had an acceptable degree of under-construction before pre-treatment. The stitch density in the course direction, for example, changed from 17 SPI to 28 SPI during pre-treatment. The resulting RPT had a stitch density that was within the acceptable range, which resulted in a desirable drapability. In this context desirable drapability indicates that the textile followed the contours provided to it and that the textile felt soft to the touch.

FIG. 10D shows an RPT after pre-treatment that was too under-constructed before pre-treatment. The stitch density in the course direction, for example, changed from 7 to 14 SPI during pre-treatment. The resulting RPT had a stitch density that was too low, which resulted in an undesirable drapability that was classified as unstructured. In this context unstructured indicates that the stitch density is too low and the contours of the material are not aesthetically pleasing.

FIG. 10E shows an RPT before and after pre-treatment that was also too under-constructed before pre-treatment. The stitch density in the course direction, for example, changed from 13 SPI to 23 SPI during pre-treatment. The resulting RPT had a stitch density that was too low, which resulted in an undesirable drapability that was classified as rope-like. In this context rope-like indicates that the textile resisted a bending force and did not want to follow contours of the body. The properties of the RPT in this sample after pre-treatment illustrate that there are a number of variables that affect the final properties of the RPT after pre-treatment, including the RPY linear density, to RPY construction, the RPT construction, and the degree of under-construction of the RPT. In the sample shown in FIG. 10E, the RPYs forming the RPT are two ended, which affected the drapability of the RPT after pre-treatment. The stitch density after pre-treatment for this sample was lower than the stitch density in the sample shown in FIG. 10C, and yet the drapability of the RPT shown in FIG. 10E was rope-like. Furthermore, the rope-like drapability of the RPT shown in FIG. 10E was similar to the drapability of the RPT shown in FIG. 10B, which had a much higher stitch density but a lower RPY linear density (it was one ended and not plied).

### ADDITIONAL CONSIDERATIONS

The foregoing description of the embodiments of the disclosure has been presented for the purpose of illustration; it is not intended to be exhaustive or to limit the claims to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the disclosure be limited not by this detailed description, but rather by any claims that issue on an application based hereon. Accordingly, the disclosure of the embodiments is intended to be illustrative, but not limiting, of the scope of the invention, which is set forth in the following claims.

What is claimed is:

1. A method of making a recombinant protein textile in a greige state before pre-treatment, comprising:
a. providing a yarn comprising a plurality of recombinant protein fibers, wherein said recombinant protein fibers supercontract upon exposure to water; and
b. generating a textile from said yarn, wherein said textile has a structure suitable for generating a pre-treated textile product with desirable properties after pre-treatment of said textile to induce supercontraction of said plurality of recombinant protein fibers.

2.-20. (canceled)

21. A method of pre-treating a textile, comprising:
a. providing a textile comprising yarn comprising a plurality of recombinant protein fibers, wherein said textile is in a greige state; and
b. exposing said textile to a solution comprising water or alcohol, wherein said exposure shrinks the textile, thereby providing a pre-treated textile.

22. The method of claim 21, further comprising placing said textile in a mold or on a 3 dimensional structure to constrain the shrinking of said textile during said pre-treatment, thereby generating a desired final textile shape.

23.-24. (canceled)

25. The method of claim 21, wherein said exposure of said textile to said solution shrinks the area of said textile by more than 20%, or more than 25%, or more than 30%, or more than 35%, or more than 40%, or more than 45%, or from 20% to 60%, or from 20% to 50%, or from 20% to 40%, or from 20% to 30%, or from 30% to 60%, or from 30% to 50%, or from 30% to 40%, or from 40% to 60%, or from 40% to 50%.

26. The method of claim 21, wherein said exposure of said textile to said solution shrinks by different amounts in the width and length, wherein the amount of shrinkage in the width, or the length, is from 5% to 50%, or from 10% to 50%, or from 15% to 50%, or from 20% to 50%, or from 25% to 50%, or from 5% to 40%, or from 10% to 40%, or from 15% to 40%, or from 20% to 40%, or from 25% to 40%.

27.-28. (canceled)

29. The method of claim 21, wherein tension is applied to said textile during said exposure of said textile to said solution.

30. The method of claim 29, wherein said tension is from 1 to 100 MPa, or from 20 to 80 MPa, or from 30 to 70 MPa, or from 40 to 60 MPa, or greater than 60 MPa, or greater than 100 MPa.

31. The method of claim 21, wherein said textile is exposed to water at a temperature at a temperature below 50° C., or below 45° C., or below 40° C., or below 35° C., or below 30° C., or is exposed to water at a temperature of from 15° C. to 50° C., or from 15° C. to 45° C., or from 15° C. to 40° C., or from 15° C. to 35° C., or from 15° C. to 30° C., or from 15° C. to 25° C., or at approximately 20° C.

32. The method of claim 21, wherein said solution is greater than 99%, greater than 90%, greater than 80%, greater than70%, or greater than 60% water or alcohol, or a mixture thereof.

33.-36. (canceled)

37. The method of claim 21, wherein said method further comprises drying said textile after said exposure of said textile to water.

38. The method of claim 37, wherein drying is performed at a temperature from 15° C. to 50° C., or from 15° C. to 45° C., or from 15° C. to 40° C., or from 15° C. to 35° C., or from 15° C. to 30° C., or from 15° C. to 25° C., or at approximately 20° C.

39. The method of claim 0, wherein tension is applied to said textile during drying.

40. (canceled)

41. The method of claim 21, wherein said pre-treated textile is resistant to shrinkage or machine washable as compared to before said pre-treatment.

42.-51. (canceled)

52. The method of claim 21, wherein said textile comprises a microknit or a microweave after said pre-treatment.

53. The method of claim 21, wherein said yarn comprises an outer sheath comprising the recombinant protein fiber, wherein the outer sheath comprises a greater twist as compared to a twist in a center core of the filament yarn; and wherein the mean dernier of the recombinant protein fiber is less than 5.

54. (canceled)

55. The method of claim 21, wherein said recombinant protein fibers comprise a recombinant silk protein.

56. The method of claim 55, wherein said recombinant silk protein comprises repeat units, wherein each repeat unit has at least 95% sequence identity to a sequence that comprises from 2 to 20 quasi-repeat units, each quasi-repeat unit having a composition comprising [GGY-{GPG-X₁]n₁-GPS-(A)n₂] (SEQ ID NO: 3), wherein for each quasi-repeat unit: X₁ is independently selected from the group consisting of SGGQQ (SEQ ID NO: 4), GAGQQ (SEQ ID NO: 5), GQGPY (SEQ ID NO: 6), AGQQ (SEQ ID NO: 7), and SQ; and n₁ is from 4 to 8, and n₂ is from 6 to 10.

57. The method of claim 55, wherein said recombinant silk protein comprises 2 or more concatenated repeats of SEQ ID NO: 1 or circularly permuted variants thereof.

58.-69. (canceled)

70. A textile comprising yarn comprising a plurality of recombinant protein fibers, wherein said textile has been supercontracted.

71.-76. (canceled)

77. The textile of claim 70, wherein said plurality of recombinant protein fibers comprise a recombinant silk protein, wherein said recombinant silk protein comprises 2 or more concatenated repeats of SEQ ID NO: 1 or circularly permuted variants thereof.

78.-79. (canceled)
